(19) United States
(12) Patent Application Publication
(10) Pub. No.: US 2009/0123551 A1
(43) Pub. Date: May 14, 2009

(54) GASTROINTESTINAL DELIVERY SYSTEMS

(75) Inventors: Elaine Phillips, San Diego, CA (US); Malcolm Hill, Solana Beach, CA (US)

Correspondence Address: WILSON SONSINI GOODRICH & ROSATI, 650 PAGE MILL ROAD, PALO ALTO, CA, 94304-1050, US

(73) Assignee: MERITAGE PHARMA, INC., San Diego, CA (US)

(21) Appl. No.: 12/269,821

(22) Filed: Nov. 12, 2008

Related U.S. Application Data
(60) Provisional application No. 61/054,105, filed on May 16, 2008.
(60) Provisional application No. 61/012,012, filed on Dec. 6, 2007.
(60) Provisional application No. 61/054,107, filed on May 16, 2008.
(60) Provisional application No. 61/015,998, filed on Dec. 21, 2007.
(60) Provisional application No. 61/019,818, filed on Jan. 8, 2008.
(60) Provisional application No. 61/034,941, filed on Mar. 7, 2008.
(60) Provisional application No. 61/054,106, filed on May 16, 2008.
(60) Provisional application No. 61/090,658, filed on Aug. 21, 2008.
(60) Provisional application No. 60/987,720, filed on Nov. 13, 2007.
(60) Provisional application No. 61/054,104, filed on May 16, 2008.

(51) Int. Cl. A61K 9/14; A61K 39/395; A61P 29/00; A61P 1/00; A61K 38/14

(52) U.S. Cl. 424/489; 424/141.1; 514/8; 977/773; 977/915

(57) ABSTRACT

Provided herein are compositions suitable for the delivery of therapeutic agents to the gastrointestinal tract. Also provided herein are methods for treating, preventing or alleviating disorders of the gastrointestinal tract, for example, those involving the esophagus, by orally administering compositions described herein.

### CROSS-REFERENCE

This application claims the benefit of U.S. Provisional Application No. 60/987,720, filed Nov. 13, 2007; U.S. Provisional Application No. 61/012,012, filed Dec. 6, 2007; U.S. Provisional Application No. 61/015,998, filed Dec. 21, 2007; U.S. Provisional Application No. 61/019,818, filed Jan. 8, 2008; U.S. Provisional Application No. 61/034,941, filed Mar. 7, 2008; U.S. Provisional Application No. 61/035,348, filed Mar. 10, 2008; U.S. Provisional Application No. 61/054,103, filed May 16, 2008; U.S. Provisional Application No. 61/054,104, filed May 16, 2008; U.S. Provisional Application No. 61/054,105, filed May 16, 2008; U.S. Provisional Application No. 61/054,106, filed May 16, 2008; U.S. Provisional Application No. 61/054,107, filed May 16, 2008; and U.S. Provisional Application No. 61/090,658, filed Aug. 20, 2008, which applications are incorporated herein by reference.

### BACKGROUND OF THE INVENTION

There are several disorders of the gastrointestinal tract, e.g., gastrointestinal inflammation, gastrointestinal cancer, gastrointestinal infection, gastrointestinal motility dysfunction, or lesions, wounds or contusions of tissue of a gastrointestinal tract. In many instances, these disorders are treated via the systemic administration of a therapeutic agent. In some instances, systemic administration of therapeutic agents leads to undesirable side effects.

### SUMMARY OF THE INVENTION

Provided in certain embodiments herein is an oral pharmaceutical composition comprising:
a. a therapeutically effective amount of one or more therapeutic agent;
b. one or more excipient that increases the interaction of the composition with a gastrointestinal surface;
c. optionally, one or more sweetener, one or more flavoring agent, or a combination thereof, and
d. one or more pharmaceutically acceptable carrier, vehicle or a combination thereof.
   In some embodiments, the oral pharmaceutical composition is physically and chemically stable. In further or alternative embodiments, the oral pharmaceutical composition is topically active.

In some embodiments, the therapeutic agent is, by way of non-limiting example, a histamine antagonist, a transient lower esophageal sphincter relaxation (TLESR) reducing agent, a prokinetic serotonergic agent, a potassium-competitive acid blocker (P-CAB), a mucosal protectant, an anti-gastrin agent, an anti-inflammatory agent, an antibacterial agent, an antifungal agent, a wound healing agent, mGluR₅ antagonists, acetylcholine modulator, 5HT₄ receptor agonist, 5HT₃ receptor antagonist, 5HT₁ receptor antagonist, an antibiotic, an antiseptic, an anesthetic, or combinations thereof.

In some embodiments, the composition remains substantially uniform after storage in ambient conditions for at least one month. In further or alternative embodiments, the composition is a solid, semi-solid, gel, cream, ointment, solution, dispersion, suspension, or paste. In further or alternative embodiments, the composition regains substantial uniformity upon mild or moderate agitation, swirling, gentle swirling or shaking. In further or alternative embodiments, the pharmaceutical composition comprises a plurality of doses and the plurality of doses are in a multiple-unit container. In further embodiments, each dose from the container of the formulation is substantially uniform with regard to each other. In further or alternative embodiments, the first and final dose from the container are substantially uniform. In further or alternative embodiments, the pharmaceutical composition comprises a plurality of particles comprising the therapeutic agent; wherein the vehicle is a liquid vehicle; and wherein the plurality of particles of the therapeutic agent are suspended in the liquid vehicle. In further or alternative embodiments, the therapeutic agent is readily dispersed throughout the composition upon mild or moderate agitation. In further or alternative embodiments, the therapeutic agent is readily suspended the composition upon mild or moderate agitation. In further or alternative embodiments, the therapeutic agent is readily re-suspended in the composition upon mild or moderate agitation after storage in ambient conditions for one month. In further or alternative embodiments, the pharmaceutical composition is a non-Newtonian fluid. In further embodiments, the non-Newtonian fluid is thixotropic. In further or alternative embodiments, the one or more excipient comprises one or more maltodextrin, dextrose, hydroxyethylcellulose (HEC), hydroxypropylmethyl-cellulose (HPMC), carboxymethyl-cellulose (CMC), microcrystalline cellulose (MCC), carbomer or combinations thereof.

In further or alternative embodiments, the therapeutic agent is topically active. In further or alternative embodiments, the therapeutic agent is a microparticle. In further or alternative embodiments, the therapeutic agent is a nanoparticle. In further or alternative embodiments, the therapeutic agent is substantially dissolved in the vehicle or carrier.

In further or alternative embodiments, the vehicle is a liquid vehicle that comprises an aqueous medium.

In further or alternative embodiments, the one or more excipient comprises CMC, and wherein the CMC is present in an amount of about 5 mg/mL to about 30 mg/mL. In further or alternative embodiments, the one or more excipient comprises carbomer, and wherein the carbomer is present in an amount of about 5 mg/mL to about 100 mg/mL. In further or alternative embodiments, the one or more excipient comprises HPMC, and wherein the HPMC is present in an amount of about 5 mg/mL to about 30 mg/mL. In further or alternative embodiments, the one or more excipient comprises MCC, and wherein the MCC is present in an amount of about 5 mg/mL to about 30 mg/mL. In further or alternative embodiments, the one or more excipient comprises a combination of CMC and MCC, wherein the CMC-MCC combination is present in an amount of about 10 mg/mL to about 40 mg/mL, and wherein the CMC/MCC mixed weight ratio is about 11/89. In further or alternative embodiments, the one or more excipient comprises dextrose, and wherein the dextrose is present in an amount of about 10 mg/mL to about 1 g/mL. In further or alternative embodiments, the one or more excipient comprises maltodextrin and wherein the maltodextrin is present in an amount of about 10 mg/mL to about 1 g/mL.

In further or alternative embodiments, the gastrointestinal surface is the esophageal epithelium (also described herein as the esophageal mucosa).

In further or alternative embodiments, any pharmaceutical composition described herein further comprises a preservative. In her embodiments, the preservative is selected from sodium benzoate, potassium sorbate, or combinations thereof. In further or alternative embodiments, any pharmaceutical composition described herein further comprises a chelating agent. In specific embodiments, the chelating agent is edetate; and wherein edetate is present in an amount of about 0.02 mg/mL to about 25 mg/mL, or about 0.05 mg/mL to about 2 mg/mL. In further or alternative embodiments, any composition described herein further comprises a buffering agent. In specific embodiments, the buffering agent comprises citrate; and wherein citrate is present in an amount of about 0.1 mg/mL to about 30 mg/mL. In further or alternative embodiments, any pharmaceutical composition described herein further comprises a surfactant. In specific embodiments, the surfactant comprises polysorbate 80; and wherein the polysorbate 80 is present in an amount of 0.05 mg/mL to about 1 mg/mL.

In further or alternative embodiments, the one or more excipient hydroxyethylcellulose (HEC), hydroxypropylmethyl-cellulose (HPMC), carboxymethyl-cellulose (CMC), microcrystalline cellulose (MCC), carbomer or a combination thereof, and the one or more excipient is present in an amount of about 5 mg/mL to about 100 mg/mL. In further or alternative embodiments, the additional excipient is, by way of non-limiting example, maltodextrin dextrose or combinations thereof and the additional excipient is present in an amount of about 1 mg/mL to about 1.5 g/mL.

In further or alternative embodiments, any pharmaceutical composition described herein has a total volume of about 1 mL to about 10 mL. In further or alternative embodiments, a singe dose of any pharmaceutical composition described herein has a total volume of about 1 mL to about 10 mL. In further or alternative embodiments, any pharmaceutical composition described herein has a total volume of about 1 mL to about 20 mL. In further or alternative embodiments, a singe dose of any pharmaceutical composition described herein has a total volume of about 1 mL to about 20 mL. In further or alternative embodiments, any pharmaceutical composition described herein has a total volume of about 7 mL or about 10 mL. In further or alternative embodiments, a singe dose of any pharmaceutical composition described herein has a total volume of about 7 mL or about 10 mL.

In further or alternative embodiments, any pharmaceutical composition described herein has a viscosity such that when a single dose of the pharmaceutical composition is orally administered to an individual, the pharmaceutical composition at least partially coats the esophagus and topically delivers an amount, e.g., a therapeutically effective amount, of therapeutic agent to a targeted site of the gastrointestinal tract, e.g., the esophagus. In further or alternative embodiments, any pharmaceutical composition described herein has a mucoadhesive characteristic such that when a single dose of the pharmaceutical composition is orally administered to an individual, the pharmaceutical composition adheres to an esophageal surface of the individual for a time sufficient to allow topical delivery of a therapeutically effective amount of the therapeutic agent to the esophagus. In some embodiments, compositions described herein are non-viscous or have low viscosity. In certain embodiments, non-viscous compositions have a viscosity of about 0.2 cP to about 5 cP, about 1 cP or about the viscosity of water. In some embodiments, low-viscous compositions have a viscosity of less than 50 cP (e.g., about 1 cP, about 1 cP to about 40 cP, about 1 cP to about 30 cP, about 1 cP to about 20 cP, about 1 cP to about 10 cP, or the like). In certain embodiments, viscosities are measured at a shear rate of about 13.2 sec⁻¹ (e.g., with a gap between the spindle and sample chamber wall of about 6.0 mm or more).

Provided in certain embodiments herein is a method of treating or alleviating symptoms of a gastrointestinal disorder, the method comprising orally administering to an individual a suitable pharmaceutical composition described herein. In some embodiments, the gastrointestinal disorder is selected from gastrointestinal inflammation, gastrointestinal cancer, gastrointestinal infection, gastrointestinal motility dysfunction, or lesions, wounds or contusions of tissue of a gastrointestinal surface. In specific embodiments, the gastrointestinal inflammation is esophageal inflammation. In further or alternative embodiments, the gastrointestinal inflammation is eosinophilic esophagitis, intermediate esophagitis, epithelial hyperplasia, basal cell hyperplasia, elongated papillae, dilated vessels in papillae, fungal esophagitis, viral esophagitis, bacterial esophagitis, corrosive esophagitis, radiation esophagitis, chemotherapy esophagitis, graft vs. host disease, a skin disease with esophageal involvement, bullous pemphigoid, pemphigus vulgaris, epidermolysis bollosa, Stevens-Johnson syndrome, Behçet's disease, sarcoidosis, idiopathic esophagitis, eosinophilic gastritis, Ménétrier's disease, parasitic gastritis, lymphocytic esophagitis, inflammatory bowel disease-associated esophagitis, parasitic gastritis, lymphocytic esophagitis, inflammatory bowel disease-associated esophagitis, tracheoesophageal fistulae, an inflammatory bowel disease involving the esophagus, reflux esophagitis, Crohn's disease, proximal gastrointestinal pathology (e.g., in individuals suffering from hypofunctioning gallbladder), eosinophilic gastrointestinal inflammation, celiac disease, eosinophilic duodenitis, duodenal eosinophilia, functional dyspepsia, esophageal inflammation secondary to caustic/irritant ingestion, persistent/recurrent esophageal strictures of any cause and including caustic/irritant ingestion, pill-induced esophagitis, systemic diseases, congenital diseases, post-surgery inflammation or gastro enteritis. In specific embodiments, the individual has eosinophilic esophagitis. In further or alternative embodiments, the gastrointestinal disorder is reflux esophagitis, gastroesophageal reflux disease (GERD), nonerosive reflux disease (NERD), Barrett's Esophagus or erosive esophagitis. In specific embodiments, the gastrointestinal cancer is esophageal cancer. In other embodiments, the gastrointestinal infection is a bacterial infection or a fungal infection. In further or alternative embodiments, the gastrointestinal motility dysfunction is esophageal motility dysfunction. In further or alternative embodiments, the lesions, wounds or contusions of tissue of a gastrointestinal surface are lesions, wounds or contusions of the esophageal epithelium.

In various embodiments, the individual is an adult, child or infant. In some embodiments, the individual is a child or infant less than 16 years old; less than 12 years old; less than 10 years old; less than 8 years old; less than 6 years old; less than 4 years old; or less than 2 years old.

Provided in some embodiments herein is a kit comprising a multiple unit container and a plurality of doses of an oral pharmaceutical composition, wherein each dose of the pharmaceutical composition comprises:
a. a therapeutically effective amount of a topically active therapeutic agent;
b. one or more excipient that increases the interaction of the composition with a gastrointestinal surface;
c. optionally one or more sweetener, one or more flavoring agent, or a combination thereof; and
d. one or more pharmaceutically acceptable carrier, vehicle or a combination thereof.

In some embodiments, the oral pharmaceutical composition is physically and chemically stable. In further or alternative embodiments, the oral pharmaceutical composition is topically active.

In further or alternative embodiments, each dose of the pharmaceutical composition within the kit further comprises a chelating agent, a surfactant, a buffering agent, and a flavoring agent. In some embodiments, the vehicle is a liquid vehicle comprising an aqueous medium. In further or alternative embodiments, the at least one additional excipient comprises one or more maltodextrin, dextrose, hydroxyethylcellulose (HEC), hydroxypropylmethyl-cellulose (HPMC), carboxymethyl-cellulose (CMC), microcrystalline cellulose (MCC), carbomer or combinations thereof. In further or alternative embodiments, the kit comprises about 10 to about 60 doses of the pharmaceutical composition. In further or alternative embodiments, the kit comprises about 150 mL of the pharmaceutical composition.

### DETAILED DESCRIPTION OF THE INVENTION

In certain embodiments, provided herein are compositions for and methods of treating, preventing or alleviating gastrointestinal disorders, including the symptoms thereof. In specific instances, the compositions and methods provided herein are useful for treating, preventing or alleviating disorders of the gastrointestinal tract. In various embodiments, gastrointestinal disorders include disorders of the upper gastrointestinal tract (e.g., the pre-colonic gastrointestinal tract). In some embodiments, the disorders of the upper gastrointestinal tract include disorders of the esophagus, stomach and/or digestive tract. In specific embodiments, the disorders of the gastrointestinal tract are disorders of the esophagus. In certain embodiments, these methods comprise orally administering to said individual a therapeutic agent (e.g., a topically active therapeutic agent) in association with at least one excipient.

An individual suitable for treatment with the compositions disclosed herein may, for example, have been diagnosed with a disorder including, but not limited to, gastrointestinal inflammation (e.g., esophageal inflammation), gastrointestinal cancer (e.g., esophageal cancer), gastrointestinal infection (e.g., bacterial or fungal), gastrointestinal motility dysfunction, or lesions, wounds or contusions of tissue of a gastrointestinal tract. The composition may also be used in treating individuals diagnosed with other gastrointestinal disorders, including stomach and duodenal ulcers, hyperactive acidic discharge disorders, such as Zollinger-Ellison syndrome and laryngeal disorders. In certain embodiments, these methods comprise orally administering to said individual a compositions described herein.

Provided herein are methods for treating, preventing and alleviating a disorder that involves the gastrointestinal tract, such as the esophagus, and responds to topical therapy. The methods and compositions of the present invention are useful, for example, for treating, preventing and alleviating gastrointestinal inflammation (e.g., esophageal inflammation), gastrointestinal cancer (e.g., esophageal cancer), gastrointestinal infection (e.g., bacterial or fungal), gastrointestinal motility dysfunction, or lesions, wounds or contusions of tissue of a gastrointestinal tract. The present methods are also useful for treating, preventing or alleviating symptoms and/or inflammation associated with other diseases or conditions of the gastrointestinal tract for example, the upper gastrointestinal tract, where it is beneficial to target a particular target site, rather than provide systemic therapy. Also provided herein are pharmaceutical compositions useful in the methods of the present application. As used herein, disorders and/or symptoms associated with a disorder disclosed herein includes inflammation and/or symptoms associated with, caused by and/or resulting from the disorder. In some embodiments, provided herein is a method of reducing cytokine and/or chemokine release in the gastrointestinal tract, such as the esophagus (e.g., in the epithelium and/or mucosa thereof) by administering a composition described herein to the gastrointestinal tract (e.g., esophagus). In certain embodiments, provided is a method of decreasing eosinophil, neutrophil and/or mast cell migration to the gastrointestinal tract (e.g., the esophagus) by administering a composition described herein to the gastrointestinal tract (e.g., the esophagus).

As used herein, unless otherwise stated, the use of the terms “a”, “an” and “the” include both singular and multiple embodiments. As used herein, the term “individual” includes any animal. In some embodiments, the animal is a mammal. In certain embodiments, the mammal is a human. In specific embodiments, the human is an adult. In other embodiments, the human is a child (e.g., a child under 12 or a child under 6). In certain embodiments, the human is an infant. As used herein, the phrase “method of treating” or “method for treating” can, in some embodiments, encompass methods of preventing, reducing the incidences of, providing prophylactic treatment, treating and alleviating. As used herein, the phrase “an effective amount” and “a therapeutically effective amount” is an amount sufficient to elicit a change in the symptoms of or a disease state of one or more gastrointestinal disorders, including but not limited to gastrointestinal inflammation (e.g., esophageal inflammation), gastrointestinal cancer (e.g., esophageal cancer), gastrointestinal infection (e.g., bacterial or fungal), gastrointestinal motility dysfunction, or lesions, wounds or contusions of tissue of a gastrointestinal tract. As used herein, the term “or” includes “and” and “or”. Treatment of a disease or disorder herein includes the treatment of the underlying causes of the disease or disorder, symptoms thereof, or the like.

As used herein, the phrase “treating inflammatory diseases involving the esophagus” includes treating symptoms of such diseases and treating inflammation associated with the diseases.

It is to be understood that any composition disclosed herein or method comprising administration of a composition disclosed herein that comprises a salt or an acid includes the disclosure of the disassociated form of the salt or acid. For example, if dissolved, sodium carboxymethylcellulose may disassociate into its sodium cationic part or parts and the corresponding carboxymethylcellulose anionic part.

Methods and Compositions

Provided herein are compositions, and methods of administering such compositions, comprising a therapeutic agent that is suitable for treating a gastrointestinal disorder. In specific embodiments, the therapeutic agent is topically active. Furthermore, provided herein are compositions for and methods of administering the therapeutic agent to the portion of the gastrointestinal tract afflicted by a gastrointestinal disorder, including a symptom of a disorder that presents in the gastrointestinal tract. As used herein, gastrointestinal disorders further include diseases, conditions and maladies of the gastrointestinal tract. In some embodiments, the therapeutic agent is an acid inhibitor (e.g., an H2 antagonist). In certain embodiments, the therapeutic agent is, by way of non-limiting example, a histamine (e.g., H1, H2, and/or H3) receptor ligand, a transient lower esophageal sphincter relaxation (TLESR)-reducing agent, a prokinetic serotonergic agent, a potassium-competitive acid blocker (P-CAB), a mucosal protectant, an anti-gastrin agent, a leukotriene antagonist, a mast cell inhibitor, a mast cell stabilizer, an immunomodulator, a biologic, an anti-asthmatic agent, a non-steroidal anti-inflammatory drug (NSAID), a chemotherapeutic, mGluR₅ antagonists, acetylcholine modulator, 5HT₄ receptor agonist, 5HT₃ receptor antagonist 5HT₁ receptor antagonist, an antibiotic, an antiseptic, an anesthetic, or a combination thereof.

In some embodiments, the therapeutic agent is an H2 receptor ligand (e.g., H2 receptor antagonist). In certain embodiments, H2 receptor antagonists useful herein include, by way of non-limiting example, cimetidine, rantitidine, famotidine and nizatidine. In some embodiments, the therapeutic agent is a H3 receptor ligand (e.g., agonist or antagonist). In certain embodiments, suitable H3 receptor agonists include, by way of non-limiting example, (R)-α-methyl-histamine. In some embodiments, the therapeutic agent is a H1 receptor ligand (e.g., antagonist).

In certain embodiments, the therapeutic agent is a TLESR-reducing agent. Suitable TLESR-reducing agents include, by way of non-limiting example, GABA_{B} agonists (e.g., baclofen), cholecystokinin (CCK-A or CCK-1) antagonists, anticholinergic agents, NO synthase inhibitors, and combinations thereof.

In some embodiments, the therapeutic agent is a prokinetic serotonergic agent. In certain embodiments, suitable prokinetic serotonergic agents include, by way of non-limiting example, 5-HT₄ receptor agonists (e.g., selective 5-HT₄ receptor agonists). 5-HT₄ receptor agonists include, by way of non-limiting example, cisapride, mosapride, tegaserod, and ATI-7505.

In some embodiments, the therapeutic agent is a potassium competitive acid blocker (P-CAB). In certain embodiments, suitable P-CAB include, by way of non-limiting example, soraprazan (BY359), revaprazan (YH1885), AZD0865, CS-526 and combinations thereof.

In certain embodiments, the therapeutic agent is a mucosal protectant. In some embodiments, suitable mucosal protectants include, by way of non-limiting example, sucralfate. In some embodiments, mucosal protectants include one or more of prostaglandin E₂ (PGE₂), epidermal growth factor (EGF) and/or transforming growth factor-α (TGF-α), or analogs thereof. In a specific embodiment, the mucosal protectant comprises the PGE₂ analog trimoprostil.

In certain embodiments, the therapeutic agent is an anti-gastrin agent. In some embodiments, suitable anti-gastrin agents include, by way of non-limiting example, cholecystokinin (CCK-B or CCK-2) antagonists. Cholecystokinin (CCK-B or CCK-2) antagonists include, by way of non-limiting example, Z-360.

In some embodiments, the therapeutic agent is a wound healing agent, an agent that promotes cell survival, an agent that promotes cell proliferation, an antifungal agent, an antibacterial agent, an antibiotic, or a combination thereof.

In further embodiments, the therapeutic agent is a promotility agent. In some embodiments, promotility agents include, by way of non-limiting embodiments, metoclopramide, cisapride, bethanechol, or the like.

In some embodiments, the therapeutic agent is a chemotherapeutic agent. In some embodiments, chemotherapeutic agents include, by way of non-limiting example, 5-fluorouracil, cisplatin, docetaxel, irinotecan, mitomycin, paclitaxel, vindesine, vinorelbine, and the like.

In certain embodiments, the therapeutic agent is a mast cell inhibitor. In some embodiments, suitable mast cell inhibitors include, by way of non-limiting example, cromolyn, cromolyn sodium, nedocromil, and the like. In certain embodiments, the therapeutic agent is a leukotriene antagonist. In some embodiments, suitable leukotriene antagonists include, by way of non-limiting example, montelukast, zafirlukast, zileuton, and the like. In some embodiments, mast cell inhibitors described herein (e.g., montelukast) are present in a composition or dose of a composition described herein in an amount sufficient to provide to an individual about 10 mg/day to about 500 mg/day, about 20 mg/day to about 40 mg/day, about 20 mg/day to about 100 mg/day, or any other therapeutically effective amount.

In some embodiments, the therapeutic agent is a non-steroidal anti-inflammatory drug (NSAID). In specific embodiments, the NSAID is ketoprofen. In various other embodiments, the therapeutic agent is an anti-inflammatory agent e.g., one as set forth in WO 2008/070129, which reference is hereby incorporated by reference in its entirety.

In some embodiments, the therapeutic agent is an immunomodulator or immunosuppressant. In specific embodiments, the immunomodulator is 6-mercaptopurine (6 MP), azathioprine, suplatast tosylate, mycophenolate mofetil, lactobacillus, calcineurin inhibitors (e.g., tacrolimus, cyclosporine, or the like), or the like.

In certain embodiments, the therapeutic agent is a biologic. In specific embodiments, the biologic is an anti IL5, an anti TNF (e.g., TNF-α), an IFN (e.g., IFN-α), an anti-eotaxin-1 antibody, an anti IL-3, an anti IgE, omalizumab, reslizumab, mepolizumab, daclizumab, SCH55700, or the like.

In some embodiments, chemotherapeutic agents include, by way of non-limiting example, imatinib, imatinib mesylate, dasatinib, AMN107, cladribine, or the like.

In some embodiments, the therapeutic agent is an antispasmodic, an agent for treating chest pain (e.g., nitrates, nitroglycerine, or the like), any drug normally administered sublingually (e.g., cardiovascular drugs, vitamins, minerals, or the like), mepoliz, esoreepraz, STI571, imatinib, an anti-CD25 (e.g., daclizumab), tyrosine kinase inhibitors, somatostatin, somatostatin analogs, an anti-CCR-3, an anti-TIM-3, ketotifen, a platelet derived growth factor receptor (PDGFR) inhibitor, or the like.

In certain embodiments, the therapeutic agent is a corticosteroid. In other embodiments, the therapeutic agent is not a corticosteroid. In some embodiments, suitable corticosteroids include, by way of non-limiting example, aclometasone, amcinonide, beclometasone, betamethasone, budesonide, ciclesonide, clobetasol, clobetasone, clocortolone, cloprednol, cortivazol, deflazacort, deoxycorticosterone, desonide desoximetasone, dexamethasone, diflorasone, diflucortolone, difluprednate, fluclorolone, fludrocortisone, fludroxycortide, flumetasone, flunisolide, fluocinolone acetonide, fluocinonide, fluocortin, fluocortolone, fluorometholone, fluperolone, fluticasone, fuprednidene, formocortal, halcinonide, halometasone, hydrocortisone aceponate, hydrocortisone buteprate, hydrocortisone butyrate, loteprednol, medrysone, meprednisone, methylprednisolone, methylprednisolone aceponate, mometasone furoate, paramethasone, prednicarbate, prednisone, prednisolone, prednylidene, remexolone, tixocortol, triamcinolone and ulobetasol, and combinations, pharmaceutically acceptable salts and esters thereof. In some embodiments, the therapeutic agent is not a corticosteroid.

In some embodiments, a daily dose of corticosteroid provided according to a method or with a composition described herein is about 0.1 mg/day to about 20 mg/day, about 0.3 mg/day to about 4 mg/day, about 0.3 mg/day to about 5 mg/day. In certain embodiments, a daily dose of corticosteroid (e.g., budesonide) provided according to a method or with a composition described herein is about 0.1 mg/day to about 20 mg/day, about 0.3 mg/day to about 4 mg/day, about 0.3 mg/day to about 3 mg/day, about 0.3 mg/day to about 1.5 mg/day, about 0.35 mg/day, about 1.4 mg/day or about 2.8 mg/day for a child aged 2-9. In certain embodiments, a daily dose of corticosteroid (e.g., budesonide) provided according to a method or with a composition described herein is about 0.1 mg/day to about 20 mg/day, about 0.3 mg/day to about 4 mg/day, about 0.3 mg/day to about 5 mg/day, about 0.5 mg/day to about 4 mg/day, about 0.5 mg/day to about 2 mg/day, about 0.5 mg/day, about 2 mg/day or about 4 mg/day for a child aged 10-17 and/or an adult. In some embodiments, a composition or dose of a composition described herein comprises about 0.1 mg/day to about 20 mg/day, about 0.3 mg/day to about 4 mg/day, about 0.3 mg to about 2.5 mg, or about 0.35 mg to about 2 mg, or about 0.35 mg, or about 0.5 mg, or about 1.4 mg, or about 2 mg of corticosteroid (e.g., budesonide).

In certain embodiments, the therapeutic agent is topically absorbed and/or adsorbed to a surface of the gastrointestinal tract (e.g., the esophagus). In some embodiments, a therapeutic agent described herein is a topically active therapeutic agent, e.g., a topically active acid inhibitor (e.g., an H2 antagonist), a topically active transient lower esophageal sphincter relaxation (TLESR)-reducing agent, a topically active prokinetic serotonergic agent, a topically active potassium-competitive acid blocker (P-CAB), a topically active mucosal protectant, a topically active anti-gastrin agent, a topically active leukotriene antagonist, a topically active mast cell inhibitor, a topically active mast cell stabilizer, a topically active immunomodulator, a topically active biologic, a topically active anti-asthmatic agent, a topically active non-steroidal anti-inflammatory drug (NSAID), a topically active chemotherapeutic, or a combination thereof.

In certain embodiments, the compositions provided herein are prepared utilizing any suitable source of active agents. In some embodiments, corticosteroid (e.g., budesonide) used in the compositions described herein are prepared with a neat therapeutic agent (e.g., a corticosteroid such as budesonide). In some embodiments, the neat agent is in a commercially available neat, bulk form. In other embodiments, the therapeutic agents are formulated as described herein from another commercially available formulation. For example, in certain embodiments compositions provided herein comprise commercially available Pulmicort Respules® (distributed by AstraZeneca, e.g., as set forth in NDA 20-929, which is hereby incorporated by reference in its entirety), Rhinocort Aqua® (distributed by AstraZeneca LP, Wilmington, Del. 19850, e.g., as set forth in NDA 20-746, which is, including all supplements, hereby incorporated herein by reference in its entirety), Symbicort® (manufactured by AstraZeneca Dunkerque Production, Dunkerque, France, e.g., as set forth in NDA 21-929, which is, including all supplements, hereby incorporated herein by reference in its entirety), or the like.

In some embodiments, the therapeutic agent is systemically absorbed through the esophagus. In specific embodiments, the systemically absorbed therapeutic agent is an agent that is degraded or loses its efficacy in some when in the stomach, e.g., a therapeutic peptide.

In certain embodiments, pharmaceutical compositions described herein provide delayed delivery of a therapeutic agent. In various embodiments, delivery of the therapeutic agent is systemic and/or local. For example, in certain embodiments, a pharmaceutical composition described herein comprises therapeutic agent and an agent for increasing the gastrointestinal transit time or decreasing the rate of gastrointestinal flow (e.g., a viscosity enhancing agent and/or mucoadhesive agent) of the composition. In some embodiments, delayed delivery of the therapeutic agent results from the increased transit time or decreased gastrointestinal flow rate of the therapeutic agent through the gastrointestinal tract, thereby delaying delivery of the composition and/or therapeutic agent to a point in the gastrointestinal tract where delivery (e.g., systemic or local) occurs (e.g., the esophagus, stomach, small intestines, and/or large intestines).

In certain instances, increased gastrointestinal transit time or decreasing the rate of gastrointestinal flow refers to the transit time or rate of flow of a composition traveling from the oral cavity (mouth) to the stomach (e.g., esophageal transit time or rate of flow along the esophagus). Similarly, in some instances, increased gastrointestinal transit time or decreasing the rate of gastrointestinal flow refers to the transit time or rate of flow of a composition traveling from the esophagus (i.e., entering the stomach) to the duodenum (e.g., stomach transit time or rate of flow in the stomach). Furthermore, in certain instances, increased gastrointestinal transit time or decreasing the rate of gastrointestinal flow refers to the transit time or rate of flow of a composition traveling from the duodenum (i.e., entering the ileum) to the ascending colon (e.g., ileum or small intestine transit time or rate of flow in the ileum or small intestine). Moreover, in some instances, increased gastrointestinal transit time or decreasing the rate of gastrointestinal flow refers to the transit time or rate of flow of a composition traveling through a combination of portions of the gastrointestinal tract, e.g., the esophagus and the stomach; the stomach and the ileum; the esophagus, the stomach and the ileum; or the like.

Furthermore, in some embodiments, provided herein is a pharmaceutical composition formulated in a dosage form for enteric delivery (e.g., in an enteric coated capsule or tablet). In certain embodiments, pharmaceutical compositions formulated in a dosage form for enteric delivery comprise a therapeutically effective amount of a therapeutic agent and an agent that increases the interaction of the composition and/or therapeutic agent with a gastrointestinal surface. In some embodiments, the pharmaceutical composition is formulated in an enteric delivery system (e.g., an enteric coated and/or delayed release pill, an enteric coated and/or delayed release capsule, an enteric coated and/or delayed release granule, an enteric coated and/or delayed release pellet an enteric coated and/or delayed release tablet, an enteric and/or delayed release matrix, a non-enteric coated viscous and/or mucoadhesive formulation, or the like). In some embodiments, enteric delivery of a pharmaceutical composition described herein allows the pharmaceutical composition to coat or at least partially coat the intestines (e.g., the small intestines, the large intestines or a combination thereof). In certain embodiments, the increased interaction of the pharmaceutical composition with the intestines, or a portion thereof allows for increased delivery of a therapeutic agent, locally and/or systemically (e.g., as compared to an otherwise similar formulation lacking the agent that increases the interaction of the pharmaceutical composition or therapeutic agent with the gastrointestinal or intestinal surface).

Provided herein are methods and pharmaceutical compositions suitable for treating, preventing or alleviating gastrointestinal disorders, including the symptoms thereof. In specific instances, the compositions and methods provided herein are useful for treating, preventing or alleviating disorders of the gastrointestinal tract and is administered, e.g., to a gastrointestinal surface (e.g., gastrointestinal mucosa, gastrointestinal epithelium, or the like). In various embodiments, gastrointestinal disorders include disorders of the upper gastrointestinal tract (e.g., the pre-colonic gastrointestinal tract). In some embodiments, the disorders of the upper gastrointestinal tract include disorders the esophagus, stomach and/or digestive tract. In specific embodiments, the disorders of the gastrointestinal tract are disorders of the esophagus. In certain embodiments, these methods comprise orally administering to said individual a therapeutic agent (e.g., a topically active therapeutic agent) in association with at least one excipient.

In some embodiments, gastrointestinal disorders treated with the methods or compositions described herein include, by way of non-limiting example, gastrointestinal inflammation (e.g., esophageal inflammation), gastrointestinal cancer (e.g., esophageal cancer), gastrointestinal infection (e.g., bacterial or fungal), gastrointestinal motility dysfunction, or lesions, wounds or contusions of tissue of a gastrointestinal tract. In specific embodiments, the gastrointestinal disorder treated with a methods or compositions described herein is an esophageal disorder including, by way of non-limiting example, esophageal inflammation, cancer of the esophagus, esophageal infection (e.g., bacterial or fungal), esophageal motility dysfunction (e.g., achalasia, diffuse esophageal spasm, and nutcracker esophagus), or lesions, wounds or contusions of tissue of an esophageal surface (which is used interchangeably herein with esophageal mucosa and esophageal epithelium). In certain embodiments, gastrointestinal inflammation (e.g., esophageal inflammation) includes, by way of non-limiting example, eosinophilic esophagitis, intermediate esophagitis (IE), epithelial hyperplasia, basal cell hyperplasia, elongated papillae, dilated vessels in papillae, fungal esophagitis, viral esophagitis, bacterial esophagitis, corrosive esophagitis, radiation esophagitis, chemotherapy esophagitis, graft vs. host disease, a skin disease with esophageal involvement, bullous pemphigoid, pemphigus vulgaris, epidermolysis bollosa, Stevens-Johnson syndrome, Behçet's disease, sarcoidosis, idiopathic esophagitis, eosinophilic gastritis, Ménétrier's disease, parasitic gastritis, lymphocytic esophagitis, inflammatory bowel disease-associated esophagitis, parasitic gastritis, lymphocytic esophagitis, inflammatory bowel disease-associated esophagitis, tracheoesophageal fistulae (TEF), reflux esophagitis, an inflammatory bowel disease involving the esophagus, Crohn's disease, proximal gastrointestinal pathology (e.g., in individuals suffering from hypofunctioning gallbladder), eosinophilic gastrointestinal inflammation, celiac disease, eosinophilic duodenitis, duodenal eosinophilia, functional dyspepsia, esophageal inflammation secondary to caustic/irritant ingestion, persistent/recurrent esophageal strictures of any cause and including caustic/irritant ingestion, pill-induced esophagitis, systemic diseases, congenital diseases, post-surgery inflammation or gastro enteritis. In specific embodiments, the gastrointestinal inflammation is eosinophilic esophagitis. In some embodiments, the gastrointestinal inflammation is reflux esophagitis, gastroesophageal reflux disease (GERD), nonerosive reflux disease (NERD), Barrett's Esophagus and/or erosive esophagitis. In some embodiments, the gastrointestinal disorder treated with the methods or compositions described herein include, by way of non-limiting example, celiac disease, drug allergy, connective tissue diseases, graft-vs-host disease, oral chronic graft-versus-host disease, radiation injury, chemical injury, oral/esophageal mucositis, oral/esophageal mucositis in cancer patients, and pharyngitis. In some embodiments, the disease treated according to a method described herein is not gastrointestinal inflammation. In certain embodiments, the disease treated according to a method described herein is not eosinophilic esophagitis.

In certain embodiments, the therapeutic agent(s) utilized herein are utilized as particles (e.g., particles comprising the therapeutic agent and suspended or dispersed in an aqueous medium). In specific embodiments, the particles are microparticles. In some embodiments, the microparticles have a mean diameter of about 0.01 microns to about 500 microns, or about 1 micron to about 500 microns. In specific embodiments, the microparticles have a mean diameter of about 1 micron to about 100 microns.

In some embodiments, a composition or formulation described herein comprises less than 50% w/w, less than 40% w/w, less than 30% w/w, less than 20% w/w, less than 10% w/w, less than 8% w/w, less than 6% w/w, less than 5% w/w, less than 4% w/w, less than 3% w/w, less than 2% w/w, or about 2% w/w, less than 1% w/w, less than 0.5% w/w, less than 0.3% w/w, less than 0.2% w/w, or about 0.2% w/w of undissolved particles. In certain embodiments, a composition or formulation described herein is substantially free of particles (i.e., undissolved particles) other than particles comprising the therapeutic agent.

In certain embodiments, pharmaceutical compositions disclosed herein and used herein comprise one or more excipients. Excipients useful herein include, by way of non-limiting example, excipients that increase the interaction of the composition and/or therapeutic agent with a gastrointestinal surface, mucoadhesive agents, viscosity enhancing agents, absorption enhancing agents, binders, fillers, lubricants, solvents, suspension agents, flavoring agents, coloring agents, sweeteners, preservatives, antioxidants, buffering agents, humectants, chelating agents, surfactants, and the like.

Excipients are used in any suitable amounts, e.g., as described herein. In certain embodiments, the pharmaceutical composition provided herein is stable. In specific embodiments, the pharmaceutical composition is chemically and/or physically stable.

Provided in certain embodiments herein are compositions or formulations comprising a therapeutic agent, one or more excipient that increases the interaction of the composition with a gastrointestinal surface (e.g., an agent that increases viscosity, mucoadhesive character, adsorption to a gastrointestinal surface, absorption of an active to and/or through a gastrointestinal surface, or combinations thereof), optionally one or more binder, optionally one or more filler, optionally one or more lubricant, optionally one or more solvent, optionally one or more suspension agent, optionally one or more flavoring agent, optionally one or more coloring agent, optionally one or more sweetener, optionally one or more preservative, optionally one or more antioxidant, optionally one or more buffering agent, optionally one or more humectant, optionally one or more chelating agent, and optionally one or more surfactant. In some embodiments, the composition described herein is a pharmaceutical composition comprising a therapeutic agent, one or more excipient that increases the interaction of the composition with a gastrointestinal surface (e.g., a gastrointestinal epithelium layer and/or a gastrointestinal mucosal layer), one or more of a flavoring agent and/or a sweetener, and one or more of a vehicle and/or a carrier. In further embodiments, the pharmaceutical composition comprises a therapeutic agent, one or more excipient that increases the interaction of the composition with a gastrointestinal surface, one or more of a flavoring agent and/or a sweetener, one or more of a vehicle and/or a carrier, a buffering agent, a surfactant, an optional chelating agent, an optional antioxidant, an optional preservative, an optional flavoring agent, at least one additional excipient, and, optionally, water.

Preservatives include, by way of non-limiting example, benzalkonium chloride, cetrimide (cetyltrimethylammonium bromide), benzoic acid, benzyl alcohol, methyl-, ethyl-, propyl- and butyl-esters of parahydroxybenzoic acid, chlorhexidine, chlorobutanol, phenylmercuric acetate, borate and nitrate, potassium sorbate, sodium benzoate, sorbic acid, thiomersal (mercurithiosalicylate), combinations thereof, or the like. Compositions and formulations described herein optionally include about 0.01% w/w to about 10% w/w, about 0.1% w/w to about 5% w/w, about 0.1% w/w to about 3% w/w, about 0.1% w/w to about 1% w/w, about 0.1% w/w to about 0.5% w/w, about 0.2% w/w of one or more preservative(s).

Antioxidants include, by way of non-limiting example, ascorbyl palmitate, butylated hydroxyanisole, butylated hydroxytoluene, monothioglycerol, sodium ascorbate, sodium formaldehyde sulfoxylate, sodium metabisulfite, BHT, BHA, sodium bisulfite, vitamin E or a derivative thereof, propyl gallate, edetate (EDTA) (e.g., disodium edetate), Diethylenetriaminepentaacetic acid (DTPA), Triglycollamate (NT), combinations thereof, or the like. Compositions and formulations described herein optionally include any suitable amount, about 0.001% w/w to about 1% w/w, about 0.01% w/w to about 0.5% w/w, about 0.01% w/w to about 0.3% w/w, or about 0.01% w/w to about 0.1% w/w one or more antioxidant(s).

Buffering agents include, by way of non-limiting example, citrate buffers (i.e., citric acid and citrate), phosphate buffers, acetate buffers, carbonate buffers (e.g., calcium carbonate, sodium bicarbonate, or the like), hydroxide (e.g., magnesium hydroxide, sodium hydroxide, or the like), combinations thereof, or the like.

Humectants include, by way of non-limiting example, glycerine, propylene glycol, ethylene glycol, glyceryl triacetate, polyols (e.g., sorbitol, xylitol, maltitol, polydextrose), and the like. Compositions and formulations described herein optionally include any suitable amount, about 0.01% w/w to about 20% w/w, about 0.1% w/w to about 10% w/w, about 1% w/w to about 10% w/w, about 1% to about 8% w/w, or about 5% w/w of a humectant. In certain embodiments, humectants inhibit or reduce precipitation and/or crystallization of one or more component of a composition or formulation described herein (e.g., a sweetener, mucoadhesive agent or a viscosity enhancing agent).

Chelating agents include, by way of non-limiting example, edetate (EDTA) (e.g., disodium edetate), Diethylenetriaminepentaacetic acid (DTPA), Triglycollamate (NT), or the like. Compositions and formulations described herein optionally include any suitable amount, about 0.001% w/w to about 3% w/w, about 0.01% w/w to about 0.5% w/w, about 0.01% w/w to about 0.3% w/w, about 0.01% w/w to about 0.1% w/w, or about 0.05% w/w of one or more chelating agent.

In certain embodiments, sweeteners include, by way of non-limiting example, glycerin, acesulfame potassium (AceK), mono-ammonium glycyrrhizinate (e.g., Magnasweet®), sucrose, lactose, glucose, fructose, arabinose, xylose, ribose, mannose, galactose, dextrose, sorbose, sorbitol, mannitol, maltose, cellobiose, xylitol and the like. In specific embodiments, the sweetener includes glycerin, acesulfame potassium and mono-ammonium glycyrrhizinate. Sweeteners are optionally included in any suitable amount including, by way of non-limiting example, about 0.01% w/w to about 60% w/w, about 0.1% w/w to about 30% w/w, about 0.1% w/w to about 5% w/w, about 5% w/w to about 20% w/w, about 0.5% w/w, about 0.8% w/w, about 1% w/w, about 6% w/w, about 7% w/w, about 8% w/w, about 9% w/w, about 10% w/w, about 11% w/w, about 12% w/w, about 13% w/w, about 14% w/w, about 15% w/w, about 16% w/w, about 17% W/W, about 18% w/w or about 19% w/w. In some embodiments, flavoring agents include, by way of non-limiting example, peppermint, orange, bubble gum, wintergreen, grape and cherry. Any suitable amount of flavoring agent is optionally utilized including, e.g., about 0.1% w/w, about 0.2% w/w, about 0.3% w/w, about 0.4% w/w, about 0.5% w/w, about 0.6% w/w, about 0.7% w/w, about 0.8% w/w, about 0.9% w/w, about 1% w/w, about 0.01% to about 5% W/W, about 0.01% w/w to about 10% w/w, or about 0.01% w/w to about 50% w/w. In certain embodiments, a composition described herein has a reduced amount of sugar sweetener (e.g., less than 20% w/w, less than 15% w/w, less than 10% W/w, less than 9% w/w, less than 8% W/W, less than 7% w/w, less than 6% w/w, less than 5% w/w, less than 4% w/w, less than 3% W/W, or less than 2% w/w) and/or a preservative to ensure stability of the composition (e.g., to reduce microbe proliferation). In specific embodiments, glycyrrhizinate such as mono-ammonium glycyrrhizinate (e.g., Magnasweet®) is present in an amount of about 0.01% w/w to about 2.95% w/w. In certain embodiments, coloring agents include yellow agents (e.g., FD&C 5 and/or 6), red agents (e.g., FD&C Red 40, Red No. 3), blue, or the like.

Surfactants include, e.g., anionic, cationic, non-ionic, or zwitterionic surfactants, such as, by way of non-limiting example, polysorbate (e.g., polysorbate 20, polysorbate 60, polysorbate 40, polysorbate 80, polysorbate 81, polysorbate 85, polysorbate 120), bile acids or their salts (e.g., sodium taurocholates, sodium deoxytaurocholates, chenodeoxycholic acid, and ursodeoxycholic acid), nonoxynol or polyoxyethylene glycol fatty acid esters, pluronic or poloxamers such as Pluronic F68, Pluronic L44, Pluronic L101, combinations thereof, or the like. In specific embodiments, the surfactant is polysorbate 80. Compositions and formulations described herein optionally include any suitable amount, e.g., about 0.001% w/w to about 10% w/w, about 0.001% w/w to about 0.5% w/w, about 0.001% w/w to about 0.3% w/w, about 0.001% w/w to about 0.1% w/w, or about 0.01% w/w of one or more surfactant.

In certain embodiments, the composition described herein is a pharmaceutical composition comprising a therapeutic agent, edetate, citrate, polysorbate 80, an optional preservative, an optional flavoring agent, one or more excipient that increases the interaction of the composition with a gastrointestinal surface, and, optionally, water. In more specific embodiments, the composition comprises water. In further or alternative embodiments, the one or more excipient that increases the interaction of the composition with a gastrointestinal surface is selected from one or more cellulose (including cellulose derivatives), dextrose, one or more maltodextrin, hydroxypropylmethyl-cellulose (HPMC), carboxymethyl-cellulose (CMC) (including, e.g., sodium carboxymethyl-cellulose (NaCMC)), microcrystalline cellulose (MCC), carbomer, hydroxyethyl cellulose (HEC), maltodextrin and combinations thereof. In a specific embodiment, the therapeutic agent is a promotility agent. In a further or additional embodiment, the one or more excipient that increases the interaction of the composition with a gastrointestinal surface comprises or is hydroxypropylmethyl-cellulose (HPMC). In a specific embodiment, the one or more excipient that increases the interaction of the composition with a gastrointestinal surface comprises or is carboxymethyl-cellulose (CMC) (including, e.g., sodium carboxymethyl-cellulose (NaCMC)). In a specific embodiment, the one or more excipient that increases the interaction of the composition with a gastrointestinal surface comprises or is microcrystalline cellulose (MCC). In a specific embodiment, the one or more excipient that increases the interaction of the composition with a gastrointestinal surface comprises or is carbomer (i.e., a high molecular weight cross-linked polyacrylic acid). In a specific embodiment, the one or more excipient that increases the interaction of the composition with a gastrointestinal surface comprises or is a combination of CMC and MCC.

In some embodiments, the one or more excipient that increases the interaction of the composition with a gastrointestinal surface is a viscosity enhancing agents. Viscosity-enhancing excipients that may be used in pharmaceutical compositions described herein include, but are not limited to, acacia (gum arabic), agar, aluminum magnesium silicate, sodium alginate, sodium stearate, bladderwrack, bentonite, carbomer, carrageenan, Carbopol, cellulose, microcrystalline cellulose (MCC), ceratonia, chondrus, dextrose, furcellaran, gelatin, Ghatti gum, guar gum, hectorite, lactose, sucrose, maltodextrin, mannitol, sorbitol, honey, maize starch, corn starch, wheat starch, rice starch, potato starch gelatin, sterculia gum, xanthum gum, polyethylene glycol (e.g. PEG 200-4500), gun tragacanth, ethyl cellulose, ethylhydroxyethyl cellulose, ethylmethyl cellulose, methyl cellulose, hydroxyethyl cellulose, hydroxyethylmethyl cellulose, hydroxypropyl cellulose, poly(hydroxyethyl methacrylate), oxypolygelatin, pectin, polygeline, povidone, propylene carbonate, methyl vinyl ether/maleic anhydride copolymer (PVM/MA), poly(methoxyethyl methacrylate), poly(methoxyethoxyethyl methacrylate), hydroxypropyl cellulose, hydroxypropylmethyl-cellulose (HPMC), carboxymethyl-cellulose (CMC), e.g., sodium carboxymethyl-cellulose NaCMC), silicon dioxide, polyvinylpyrrolidone (PVP: povidone), Splenda® (dextrose, maltodextrin and sucralose) or combinations thereof. In one non-limiting example, the viscosity-enhancing excipient is Splenda®. In specific embodiments, the viscosity-enhancing excipient is a combination of MCC and CMC (e.g., Avicel RC-591). In some embodiments, the CMC/MCC combination (e.g., Avicel RC-591) is present in the composition in an amount of about 1 mg/mL to about 150 mg/mL, 1 mg/mL to about 75 mg/mL, or about 5 mg/mL to about 40 mg/mL. In certain embodiments, the CMC/MCC mixed weight ratio is between about 1/99 and about 99/1, about 20/80 and about 5/95, or about 15/85 and about 10/90. In a specific embodiment, the CMC is NaCMC and the CMC/MCC mixed weight ratio is about 11/89. In a specific embodiment, the viscosity enhancing agent is selected from, by way of non-limiting example, Carbopol 974P, Carbopol Ultrez 10, sodium alginate LF120 and sodium alginate H120L. In some embodiments, the viscosity enhancing agent is selected from, by way of non-limiting example, PVP 10,000, PEG 3350 and HiFibro.

In certain embodiments, suitable viscosity enhancing agents include, by way of non-limiting embodiment, one or more cellulose (including cellulose derivatives), dextrose, one or more maltodextrin hydroxypropylmethyl-cellulose (HPMC), carboxymethyl-cellulose (CMC) (including, e.g., sodium carboxymethyl-cellulose (NaCMC)), microcrystalline cellulose (MCC), carbomer, hydroxyethyl cellulose (HEC), maltodextrin and combinations thereof. In a further or additional embodiment, the viscosity enhancing agent comprises or is hydroxypropylmethyl-cellulose (HPMC). In a specific embodiment, the viscosity enhancing agent comprises or is carboxymethyl-cellulose (CMC) (including, e.g., sodium carboxymethyl-cellulose (NaCMC)). In a specific embodiment, the viscosity enhancing agent comprises or is microcrystalline cellulose (MCC). In a specific embodiment, the viscosity enhancing agent comprises or is carbomer (i.e., a high molecular weight cross-linked polyacrylic acid). In a specific embodiment, the viscosity enhancing agent comprises or is a combination of CMC and MCC.

In specific embodiments, the one or more excipient that increases the interaction of the composition with a gastrointestinal surface comprises or is a mucoadhesive agent. As used herein, mucoadhesive agents are agents that increase the interaction of a composition with a surface of the gastrointestinal tract (e.g., the mucosa and/or epithelium of the gastrointestinal tract or a specific site of the gastrointestinal tract, such as the esophagus). In some embodiments, suitable mucoadhesive agents include, by way of non-limiting example, maltodextrin.

Mucoadhesive agents to be used herein include, by way of non-limiting example, a soluble polyvinylpyrrolidone polymer (PVP), a cadherin (e.g., c-Cad), a carbopol, a crosslinked poly(acrylic acid) (e.g., Carbopol 974P), a carbomer homopolymer, a carbomer copolymer, a water-swellable, but water-insoluble, fibrous, cross-linked carboxy-functional polymer, a hydrophilic polysaccharide gum, one or more maltodextrin, alginate, a cross-linked aliginate gum gel, thiomers (e.g., thiolated chitosan, thiolated polycarbophil, thiolated alginate, thiolated cellulose derivatives, thiolated carboxymethyl cellulose, thiolated polyacrylic acid, or thiolated polyacrylates), PEGylated polymers (e.g., PEGylated polyacrylic acid or PEGylated polyacrylates), lectin, hydroxypropyl methyl cellulose (HPMC), cellulose derivatives, HPMA copolymers, a water-dispersible polycarboxylated vinyl polymer. In some embodiments, the mucoadhesive agent is a carbopol. In a specific embodiment, the mucoadhesive agent is selected from, by way of non-limiting example, Carbopol 974P, Carbopol Ultrez 10, sodium alginate LF120 and sodium alginate H120L. As used herein, a mucoadhesive agent is an agent that adheres to a gastrointestinal surface (e.g., either or both of a gastrointestinal epithelia or mucosa).

In some embodiments, the one or more excipient that increases the interaction of the composition with a gastrointestinal surface comprises at least one maltodextrin. In certain embodiments, the excipient that increases the interaction of the composition with a gastrointestinal surface (e.g., maltodextrin, CMC, MCC, or a combination thereof) is completely, substantially, or at least partially dissolved in a liquid vehicle. In some embodiments, any composition or formulation described herein comprises a first excipient that increases the interaction of the composition with a gastrointestinal surface (e.g., maltodextrin) that is substantially or at least partially dissolved in a liquid vehicle (or substantially soluble in saliva when orally administered) and a second excipient that increases the interaction of the composition with a gastrointestinal surface that is substantially insoluble in a liquid vehicle (or in saliva when orally administered). In some embodiments, an oral pharmaceutical composition described herein comprises less than about 0.1 g or less than about 1 g of maltodextrin for every mL of liquid vehicle in the oral pharmaceutical composition. In certain instances, a composition or formulation described herein comprises less than 2 g of maltodextrin/mL of composition, less than 1.5 g of maltodextrin/mL of composition, less than 1 g of maltodextrin/mL of composition, less than 0.5 g of maltodextrin/mL of composition, less than 0.25 g/mL of maltodextrin/mL of composition, about 0.05 g of maltodextrin/mL of composition to about 0.5 g of maltodextrin/mL of composition, about 0.05 g of maltodextrin/mL of composition to about 0.4 g of maltodextrin/mL of composition, about 0.05 g of maltodextrin/mL of composition to about 0.3 g of maltodextrin/mL of composition, about 0.1 g of maltodextrin/ml of composition to about 0.5 g of maltodextrin/mL of composition, about 0.1 g of maltodextrin/mL of composition to about 0.4 g of maltodextrin/mL of composition, about 0.1 g of maltodextrin/mL of composition to about 0.3 g of maltodextrin/mL of composition, about 0.2 g of maltodextrin/mL of composition to about 0.5 g of maltodextrin/mL of composition, about 0.2 g of maltodextrin/mL of composition to about 0.4 g of maltodextrin/mL of composition, or about 0.2 g of maltodextrin/mL of composition to about 0.3 g of maltodextrin/mL of composition. In some embodiments, any composition or formulation described herein comprises greater than about 7% w/w, greater than about 8% w/w, greater than about 9% w/w, greater than about 10% w/w, greater than about 11% w/w, greater than about 12% w/w, greater than about 13% w/w, greater than about 14% w/w, greater than about 15% w/w, greater than about 16% w/w, greater than about 17% w/w, greater than about 18% w/w, greater than about 19% w/w, greater than about 20% w/w, greater than about 21% w/w, greater than about 22% w/w, greater than about 23% w/w, greater than about 24% w/w, greater than about 25% w/w, greater than about 26% w/w, greater than about 27% w/w, greater than about 28% w/w, greater than about 29% w/w or greater than about 30% w/w of maltodextrin (as used herein greater than includes, e.g., up to about 75% w/w, about 80% w/w, about 90% w/w, about 95% w/w, about 98% w/w, or about 99% w/w). In some embodiments, the maltodextrin is substantially dissolved in the liquid vehicle. In certain embodiments, the maltodextrin has a dextrose equivalents (DE) of greater than 4, greater than 5, greater than 10, greater than 11, greater than 12, greater than 13, greater than 14, greater than 15, about 15, about 4 to about 10, about 4 to about 9, about 4 to about 8, about 11 to about 20, about 12 to about 19, about 13 to about 18, or about 14 to about 16. In some embodiments, a composition described herein comprises a first maltodextrin and a second maltodextrin. In specific embodiments, the first maltodextrin has a DE of about 4 to about 10, about 4 to about 9, or about 4 to about 8 and the second maltodextrin has a DE of about 10 to about 20, about 12 to about 19, or about 13 to about 18. Exemplary maltodextrins include, by way of non-limiting example, Maltrin® M040 (with an average DE of about 5), Maltrin® M050 (with an average DE of about 5), Maltrin® M100 (with an average DE of about 10), Maltrin® M150 (with an average DE of about 15), Maltrin® M180 (with an average DE of about 18), Maltrin® M440 (with an average DE of about 5), Maltrin® M500 (with an average DE of about 10), Maltrin® M510 (with an average DE of about 10), Maltrin® M550 (with an average DE of about 15), Maltrin® M580 (with an average DE of about 18), Maltrin® M700 (with an average DE of about 10), C*Pharm® maltodextrins (with average DE values of about 7, about 14, 18.5, etc.), or the like.

In some embodiments, at least one maltodextrin utilized in a composition described herein has a molecular weight high enough to increase the solubility of a therapeutic agent, or to increase the suspendability (including, e.g., stability of a suspension, amount of time, effort and/or agitation required to make a suspension or stable suspension, sedimentation rate of the suspension, or a combination thereof) of a plurality of particles comprising a therapeutic agent. In certain embodiments, the DE and/or degree of branching of the maltodextrin is selected in order to achieve increased solubility of the therapeutic agent. In certain embodiment, the maltodextrin increases the solubility of a therapeutic agent in an aqueous vehicle, wherein the therapeutic agent has sparing solubility in water. In some embodiment, the maltodextrin increases the suspendability of a plurality of particles comprising a therapeutic agent in an aqueous vehicle, wherein the therapeutic agent has sparing solubility in water. In some instances, for example, about 500 μg/mL or less, about 250 μg/mL or less, about 200 μg/mL or less, about 150 μg/mL or less, about 100 μg/mL or less, about 75 μg/mL or less, or about 50 μg/mL or less of a therapeutic agent with sparing solubility dissolves in water (e.g., deionized or neutral water) under ambient conditions.

As used herein, “edetate” includes all compounds of Formula I wherein each R is independently selected from an H and a negative charge (e.g., as a salt or as a disassociated salt or acid). In certain embodiments, edetate is selected from, by way of non-limiting example, disodium edetate, calcium edetate, ethylenediaminetetraacetic acid and the like.

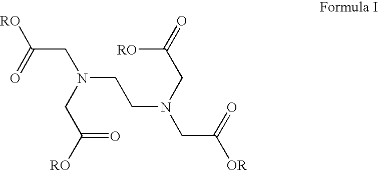

As used herein, “citrate” includes all compounds of Formula II wherein each R is independently selected from an H and a negative charge (e.g., as a salt or as a disassociated salt or acid). In certain embodiments, citrate is selected from, by way of non-limiting example, sodium citrate, citric acid and the like.

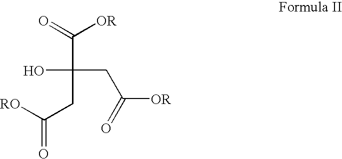

In certain embodiments, sweeteners include, by way of non-limiting example, sucrose, lactose, glucose, fructose, arabinose, xylose, ribose, mannose, galactose, dextrose, sorbose, sorbitol, mannitol, maltose, cellobiose, xylitol and the like. In some embodiments, flavoring agents include, by way of non-limiting example, peppermint, orange, bubble gum, wintergreen, grape and cherry.

Preservatives include, by way of non-limiting example, benzalkonium chloride, methylparaben (e.g., sodium methylparaben), propylparaben, potassium sorbate and sodium benzoate. In specific embodiments, the preservative is potassium sorbate.

In certain embodiments, a composition described herein comprises an active, a commercially available formulation, and, optionally, one or more additional excipient. In some embodiments, a composition described herein comprises an active formulated in a manner similar to a commercial formulation (e.g., lacking one or more of the active ingredients of the formulation), and, optionally, one or more additional excipient. The one or more additional excipients can be utilized to achieve a formulation as described herein. In specific embodiments, the commercially available formulation is Ultra XCID (manufactured by Matrixx Initiatives, Inc., Phoenix, Ariz.).

In certain embodiments, a composition provided herein is prepared by combining the components set forth in any of Tables 1-4. In various embodiments, one or more of maltodextrin, dextrose, HEC, CMC, MCC, Carbomer and HPMC are utilized therein.

TABLE 1
Composition #1

| Ingredient | Amount |
|---|---|
| Therapeutic Agent | 1 mg to 1 g |
| CMC, MCC, Carbomer, HPMC and/or HEC | 0.5 g to 10 g |
| Dextrose | 0 g to 100 g |
| Maltodextrin | 0 g to 100 g |
| Disodium Edetate | 5 mg to 200 mg |
| Citric Acid | 10 mg to 1 g |
| Sodium Citrate | 10 mg to 2 g |
| Tween 80 | 5 mg to 100 mg |
| Flavoring Agent | optional |
| Sweetener | optional |
| Preservative | optional |
| Water | q.s. to 100 mL |

TABLE 2
Composition #2

| Ingredient | Amount |
|---|---|
| Therapeutic Agent | 1 mg to 1 g |
| CMC, MCC, Carbomer, HPMC and/or HEC | 0 g to 10 g |
| Dextrose | 1 g to 100 g |
| Maltodextrin | 0 g to 100 g |
| Disodium Edetate | 5 mg to 200 mg |
| Citric Acid | 10 mg to 1 g |
| Sodium Citrate | 10 mg to 2 g |
| Tween 80 | 5 mg to 100 mg |
| Flavoring Agent | optional |
| Sweetener | optional |
| Preservative | optional |
| Water | q.s. to 100 mL |

TABLE 3
Composition #3

| Ingredient | Amount |
|---|---|
| Therapeutic Agent | 1 mg to 1 g |
| CMC, MCC, Carbomer, HPMC and/or HEC | 0 g to 10 g |
| Dextrose | 0 g to 100 g |
| Maltodextrin | 1 g to 100 g |
| Disodium Edetate | 5 mg to 200 mg |
| Citric Acid | 10 mg to 1 g |
| Sodium Citrate | 10 mg to 2 g |
| Tween 80 | 5 mg to 100 mg |
| Flavoring Agent | optional |
| Sweetener | optional |
| Preservative | optional |
| Water | q.s. to 100 mL |

TABLE 4
Composition #4

| Ingredient | Amount % w/w |
|---|---|
| Therapeutic agent | 0.001 to 30 |
| Sodium methylparaben | 0.0001 to 0.1 |
| Sorbitol | 5 to 30 |
| Sucrose | 1 to 40 |
| Corn starch | 1 to 10 |
| MCC | 0.1 to 5 |
| CMC (NaCMC) | 0.1 to 5 |
| Xanthan | 0.001 to 1 |
| Glycerin | 0.1 to 10 |
| Calcium carbonate | 0 to 30 |
| Magnesium hydroxide | 0 to 5 |
| Color (e.g., FD&C Red No. 3) | optional |
| Water | q.s. to 1, 2, 3, 4, 5, 6, 7, 8, 9, |
| | 10, 11, 12, 13, 14, or 15 mL |

In certain embodiments, the excipient or excipients chosen increase the interaction of the composition with the surface of the gastrointestinal tract (e.g., the mucosa and/or epithelium of the gastrointestinal tract or of a specific site of the gastrointestinal tract, such as the esophagus) by at least 1.02 fold, by at least 1.05-fold, by at least 1.1 fold, by at least 1.2 fold, by at least 1.25-fold, by at least 1.5-fold, by at least 2-fold, by at least 3-fold, by at least 4-fold or by at least 5-fold. In certain embodiments, the increased interaction of the composition is an at least 1.02 fold, by at least 1.05-fold, by at least 1.1 fold, by at least 1.2 fold, by at least 1.25-fold, by at least 1.5-fold, by at least 2-fold, by at least 3-fold, by at least 4-fold or by at least 5-fold of interaction of the composition with the esophagus that occurs following passing of the bolus of the composition being swallowed (e.g., 5 seconds, 6 seconds, 7 seconds, 8 seconds, 9 seconds, 10 seconds, 11 seconds, 12 seconds, 13 seconds, 14 seconds, 15 seconds, or the like after initial swallowing of the composition). In certain embodiments, these increases are measured and compared to the measure of an otherwise similar composition lacking the excipient or excipients that increase the interaction of the composition with the surface of the gastrointestinal tract. In certain instances, increased interaction of the composition is measured as a function of the amount of composition present in the esophagus (e.g., after the bolus has passed through the esophagus). In specific instances, the amount of composition present in the esophagus is measured in any suitable manner, e.g., by radiolabeling the composition and measuring the amount of the composition in the esophagus utilizing gamma scintigraphy. An increase in the interaction of the composition with the surface of the gastrointestinal tract (e.g., the surface of the esophagus) may be measured by measuring the retention time of the material along a length of a surface of the gastrointestinal tract (e.g., the surface of the esophagus), wherein the retention time is increased in the presence of the excipients as compared to its absence. In another embodiment, an increased interaction may be measured by the decrease in physiological manifestations or symptoms of the disease or ailment to be treated, including a decrease in total eosinophil counts in a target sample.

In specific embodiments, following oral administration of a composition described herein to the esophagus (e.g., following initial swallowing or drinking of the composition), at least 1%, 2%, 3%, 4%, 5%, 6%, 7%, 8%, 9%, 10%, 15%, 20%, 30%, 40%, 50%, 60%, 70%, 80%, 90% or 95% by weight of the corticosteroid or composition administered is present within the esophagus (e.g., as measured by gamma scintigraphy) after at least 5 seconds, 10 seconds, 15 seconds, 20 seconds, 25 seconds, 30 seconds, 40 seconds, 45 seconds, 50 seconds, or 1, 1.5, 2, 3, 4, 5, 6, 7, 8, 9, 10, 15, 20, 25, 30, 35, 40, 45, 50, 55 or 60 minutes following application of the composition to the esophagus. In certain instances, even small differences (e.g., increases) in adherence times (e.g., residence times) between formulations can result in therapeutically significant or clinically significant results or improvements.

In some embodiments, any pharmaceutical composition described herein is stable. In specific embodiments, the pharmaceutical composition is chemically and physically stable. In certain embodiments, chemical stability is evidenced by a pharmaceutical composition that comprises at least 80%, 90%, 95%, 98%, or 99% of the initial amount or label amount of therapeutic agent therein for, by way of non-limiting example, 1 week, 2 weeks, 3 weeks, 1 month, 3 months, 6 months, 1 year, 2 years, or for the duration of the shelf life. In some embodiments, physical stability is evidenced by a pharmaceutical composition that is able to substantially obtain uniformity, remain substantially uniform (e.g., for at least 1 day, 3 days, 1 week, 2 weeks, 3 weeks, 1 month, 3 months, 6 months, 1 year, 2 years, etc.), or substantially regain uniformity (e.g., via mild or moderate agitation after being undisturbed for 1 day, 3 days, 1 week, 2 weeks, 3 weeks, 1 month, 3 months, 6 months, 1 year, 2 years, etc.). In certain embodiments, uniformity as described herein is evidenced by the uniformity of the dispersion of the particles (e.g., particles comprising a therapeutic agent and/or an excipient) throughout the pharmaceutical composition, the uniformity of the dispersed mass of therapeutic agent throughout the pharmaceutical composition, the uniformity of the concentration of one or more of the components in the composition throughout the pharmaceutical composition, and the like. In some embodiments, the uniformity of the dispersed mass or therapeutic agent throughout the pharmaceutical composition is obtained through mild or moderate agitation and such uniformity is retained for a convenient amount of time following the agitation (e.g., for a time sufficient to allow administration of a therapeutically effective dose from the composition). In certain embodiments, mild or moderate agitation includes, by way of non-limiting example, shaking, shaking well, swirling, gentle swirling, and the like. In some embodiments, mild or moderate agitation includes agitation without a special apparatus. In some embodiments, uniformity of the pharmaceutical composition refers to dose uniformity (e.g., each dose delivered or withdrawn from the composition comprises a substantially similar amount of therapeutic agent), or the concentration of therapeutic agent in at least some or all of the doses from the multiple dose formulations are substantially similar. In certain embodiments, substantially similar includes, e.g., within 20%, 15%, 10%, 7%, 5%, 3%, 2%, or 1%. In some embodiments, uniformity of the pharmaceutical composition refers to uniformity of the interaction of each dose of the pharmaceutical composition with a gastrointestinal (e.g., esophageal) surface. In specific instances, the uniformity of the interaction includes, e.g., degree and/or time of adhesion to a gastrointestinal (e.g., esophageal) surface, time of residence upon a gastrointestinal (e.g., esophageal) surface, and/or amount of therapeutic agent absorbed by a gastrointestinal (e.g., esophageal) surface. In certain embodiments, physical stability is evidenced by a composition that comprises at least 80%, 90%, 95%, 98%, or 99% of the initial amount or label amount of active or actives therein for, by way of non-limiting example, 2 days, 1 week, 2 weeks, 3 weeks, 1 month, 3 months, 6 months, 1 year, 2 years, or for the duration of the shelf life.

In some embodiments, a pharmaceutical composition described herein comprises a therapeutic agent (as a solute, a particle, or a combinations thereof) that is readily dispersed throughout the composition upon agitation (e.g., mild or moderate). In certain embodiments, a pharmaceutical composition described herein comprises a therapeutic agent that is readily dispersed throughout the composition upon mild or moderate agitation after storage. In some embodiments, a pharmaceutical composition described herein comprises a plurality of particles comprising a therapeutic agent that is easily re-suspended in the composition upon mild or moderate agitation. In certain embodiments, a pharmaceutical composition described herein comprises a plurality of particles comprising a therapeutic agent that is easily re-suspended in the composition upon mild or moderate agitation after storage. In some embodiments, a pharmaceutical composition described herein comprises a plurality of particles comprising a therapeutic agent that is readily dispersed throughout the composition upon mild or moderate agitation. In certain embodiments, a pharmaceutical composition described herein comprises a plurality of particles comprising a therapeutic agent that is readily dispersed throughout the composition upon mild or moderate agitation after storage. In certain embodiments, particles comprising a therapeutic agent are able to be easily re-suspended and/or readily re-dispersed in a pharmaceutical composition described herein after storage under ambient conditions. In other embodiments, the storage is under an inert atmosphere, increased temperature and/or increased relative humidity. As used herein, the pharmaceutical compositions described herein have one or more of the properties described herein after storage for, by way of non-limiting example, 1 day, 2 days, 3 days, 1 week, 2 weeks, 3 weeks, 1 month, 3 months, 6 months, 1 year, 2 years, or the time of the shelf life.

In some embodiments, any pharmaceutical composition described herein optionally comprises a small amount of glycine, e.g., an amount that does not result in a substantial change in viscosity of the composition.

In some embodiments, a composition described herein adheres to or resides upon the esophagus, or some portion thereof, after oral administration for at least 6 seconds, for at least 12 seconds, for at least 15 seconds, for at least 30 seconds, for at least 60 seconds, for at least 90 seconds, for at least 120 seconds, for at least 3 minutes, for at least 4 minutes, for at least 5 minutes, for at least 15 minutes, or for at least 30 minutes. In certain embodiments, the composition is retained on the esophagus after oral administration for about 15 seconds to about 120 seconds, or for about 30 to about 90 seconds. In some embodiments, a portion of the composition comprises about 90% or more, about 80% or more, about 70% or more, about 60% or more, about 50% or more, about 40% or more, about 30% or more, about 20% or more, about 10% or more, or about 5% or more. In some embodiments, when an oral pharmaceutical composition described herein is administered to an esophagus, e.g., by oral administration, at least 50%, 40%, 30%, 20%, 15%, 10%, 9%, 8%, 7%, 6%, 5%, 4%, 3%, 2%, or 1% of the oral pharmaceutical composition adheres to or resides upon/within the esophagus for at least 5 seconds, 10 seconds, 15 seconds, 30 seconds, 45 seconds, 1 minute, 2 minutes, 3 minutes, 5 minutes, 10 minutes, 15 minutes, 20 minutes, 30 minutes, 45 minutes, 1 hour, or more. In certain embodiments, when an oral pharmaceutical composition described herein is administered to the esophagus, e.g., by oral administration, at least 50%, 40%, 30%, 20%, 15%, 10%, 9%, 8%, 7%, 6%, 5%, 4%, 3%, 2%, or 1% of the corticosteroid adheres to or resides upon the esophagus for at least 5 seconds, 10 seconds, 15 seconds, 30 seconds, 45 seconds, 1 minute, 2 minutes, 3 minutes, 5 minutes, 10 minutes, 15 minutes, 20 minutes, 30 minutes, 45 minutes, 1 hour, or more. In some embodiments, at least 50%, 40%, 30%, 20%, 15%, 10%, 9%, 8%, 7%, 6%, 5%, 4%, 3%, 2%, or 1% of the corticosteroid adheres to, resides in/on, or is absorbed by the esophagus at least 5 seconds, 10 seconds, 15 seconds, 30 seconds, 45 seconds, 1 minute, 2 minutes, 3 minutes, 5 minutes, 10 minutes, 15 minutes, 20 minutes, 30 minutes, 45 minutes, 1 hour, or more after application of an oral pharmaceutical composition described herein is administered to the esophagus, e.g., by oral administration (e.g., swallowing). In certain embodiments, administration of the oral pharmaceutical composition to the esophagus includes orally administering and/or swallowing at least part of the oral pharmaceutical composition or dose of the oral pharmaceutical composition.

In certain embodiments, adherence and/or absorption of a pharmaceutical composition or corticosteroid described herein to a gastrointestinal mucosal site (e.g., esophagus) may be determined in any suitable manner, e.g., by scintigraphy or by an assay. In some embodiments, such determinations are performed in vivo or in vitro. In certain embodiments, in vivo scintigraphy may include combining a pharmaceutical composition described herein with a detectable radioisotope, administering the labeled composition to a subject and detecting and/or measuring the adherence or residence of the pharmaceutical composition or corticosteroid to the gastrointestinal surface (e.g., esophagus) with a device (e.g., camera) that detects and/or measures radioactivity. In some embodiments, in vivo scintigraphy may include linking a corticosteroid described herein with a detectable radioisotope, formulating the labeled corticosteroid into a composition described herein, administering the composition to a subject and detecting and/or measuring the adherence or residence of the pharmaceutical composition or corticosteroid to the gastrointestinal surface (e.g., esophagus) with a device (e.g., camera) that detects and/or measures radioactivity. In certain embodiments, an in vitro assay for detecting adherence of a pharmaceutical composition or corticosteroid described herein to a gastrointestinal mucosal site (e.g., esophagus) may include applying a composition described herein to a distal portion of a strip of gastrointestinal mucosal tissue (e.g., porcine esophageal tissue) and subjecting the composition to a flow of artificial saliva in the direction of the opposite distal portion of the strip. Determination of adherence or residence of the composition and/or corticosteroid may be determined at a given time by detecting either the amount of composition and/or corticosteroid eluted or the amount of composition and/or corticosteroid remaining on the gastrointestinal surface (e.g., esophagus).

In certain embodiments, a composition described has a viscosity sufficient to deliver an effective amount of the composition to the site of gastrointestinal affliction, e.g., the esophagus. In some embodiments, the effective amount of the composition delivered to the esophagus is an amount sufficient to coat, or partially coat, the esophagus, and deliver the composition to the affected areas, including by way of example only, a portion of the esophagus, the lower esophagus, the esophageal-stomach juncture, the stomach and/or the duodenum. In certain embodiments, the viscosity of the oral dosage form is such that when administered orally, it is not so thick as to cause difficulty in swallowing, cause gagging, and/or be unpalatable. In certain embodiments, the viscosity of the oral dosage form is a viscosity that is sufficient to provide exposure of the therapeutic agent to the esophagus for a sufficient period of time such that the disorder or symptoms of the disorder involving the gastrointestinal tract, including the esophagus, are reduced following administration of a pharmaceutical composition described herein formulated in or as an oral dosage form. In some instances wherein the pharmaceutical composition is in solid or semi-solid form, the composition described has a viscosity, when mixed with saliva or water (e.g., in certain instances wherein the composition is formulated as an effervescent tablet, sachet or home brew), the resulting composition has a viscosity sufficient to deliver an effective amount of a composition described herein to the site of gastrointestinal inflammation, e.g., the esophagus.

Viscosity may be, for example, measured at room temperature, at about 20-25 degrees Celsius, or at about 37 degrees Celsius to mimic body temperature. In various embodiments of the present invention, the viscosity of the composition described herein is any viscosity suitable for delivery of the therapeutic agent to the targeted and/or afflicted portion of the gastrointestinal tract. In some embodiments, the viscosity of the composition is at least about 1 or 2 centipoise (cP), at least about 3 cP, at least about 5 cP, at least about 10 cP, at least about 15 cP, at least about 20 cP, at least about 25 cP, at least about 30 cP, at least about 35 cP, at least about 40 cP, at least about 50 cP, at least about 200 cP, or at least about 225 cP. In some embodiments, the viscosity of the composition is at least about 100 cP. In certain embodiments, the viscosity of the composition, measured at 25 degrees Celsius, is about 50 cP to about 250,000 cP, about 50 cP to about 70,000 cP, about 50 cP to about 25,000 cP, about 50 cP to about 10,000 cP, about 50 cP to about 3,000 cP, or about 50 cP to about 2,000 cP. In one aspect, the viscosity of the composition, as measured at 25 degrees Celsius, is from about 25 centipoise (cP) to about 800 cP, about 50 cP to about 800, or about 300 cP to about 800 cP (e.g., measured by a Brookfield viscometer). In another aspect, the viscosity of the composition may range from about 100 cP to about 200 cP, about 200 cP to about 300 cP, about 250 cP to about 600 cP or about 400 cP to about 600 cP. In specific embodiments, the viscosity of the formulation is about 30 cP, about 100 cP, about 200 cP, about 300 cP, about 400 cP, about 500 cP, or about 250,000 cP (e.g., as measured with a Brookfield viscometer at 25 degrees Celsius equipped with an ultra low adapter).

In some embodiments, the viscosity of the composition is measured at room temperature (about 25 degrees C.) with a shear rate of about 13.2 sec⁻¹ (e.g., with gap between the spindle and sample chamber wall of about 6.0 mm). In certain embodiments, provided herein is a composition having a viscosity under such conditions that is at least about 1 cP, at least about 2 cP, at least about 3 cP, at least about 5 cP, at least about 10 cP, at least about 15 cP, at least about 20 cP, at least about 25 cP, at least about 25 centipoise (cP), at least about 30 cP, at least about 35 cP, at least about 40 cP, at least about 50 cP, at least about 200 cP, at least about 225 cP, at least about 250 cP, at least about 300 cP, or at least about 400 cP. In some embodiments, the viscosity of the composition under such conditions is about 50 cP to about 250,000 cP, about 50 cP to about 70,000 cP, about 50 cP to about 25,000 cP, about 50 cP to about 10,000 cP, about 50 cP to about 3,000 cP, about 50 cP to about 2,000 cP, about 250 cP to about 250,000 cP, about 250 cP to about 70,000 cP, about 250 cP to about 25,000 cP, about 250 cP to about 10,000 cP, about 250 cP to about 3,000 cP, or about 250 cP to about 2,000 cP. In one aspect, the viscosity of the composition, as measured at 25 degrees Celsius, is from about 25 centipoise (cP) to about 800 cP, about 50 cP to about 800, or about 300 cP to about 800 cP (e.g., measured by a Brookfield viscometer). In another aspect, the viscosity of the composition under such conditions may range from about 100 cP to about 200 cP, about 200 cP to about 300 cP, about 250 cP to about 600 cP or about 400 cP to about 600 cP. In specific embodiments, the viscosity of the formulation measured under such conditions is about 30 cP, about 40 cP, about 100 cP, about 200 cP, about 300 cP, about 400 cP, about 500 cP, or about 250,000 cP. In some embodiments, compositions described herein have a shear of greater than 150 cP, greater than 160 cP, greater than 170 cP, greater than 180 cP, or greater than 190 cP at a shear rate of 15 sec⁻, 16 sec⁻¹, 17 sec⁻¹, 18 sec⁻¹, 19 sec⁻¹, or 20 sec⁻¹.

In some embodiments, the viscosity of the composition is measured at room temperature (about 25 degrees C.) with a shear rate of about 15 sec⁻¹ (e.g., with a gap between the spindle and the sample chamber wall of about 6 mm or greater). In certain embodiments, provided herein is a composition having a viscosity under such conditions that is at least about 2 cP, at least about 5 cP, at least about 10 cP, at least about 15 cP, at least about 20 cP, at least about 25 cP, at least about 50 cP, at least about 100 cP, at least about 150 centipoise (cP), at least about 160 cP, at least about 170 cP, at least about 180 cP, at least about 190 cP, or at least about 200 cP. In some embodiments, the viscosity of the composition under such conditions is about 150 cP to about 250,000 cP, 160 cP to about 250,000 cP, 170 cP to about 250,000 cP, 180 cP to about 250,000 cP, or 190 cP to about 250,000 cP. Viscosity can also be determined by any method that will measure the resistance to shear offered by the substance or preparation. Many viscometers are available to those in the pharmaceutical field, and include those built by, for example, Brookfield.

In some embodiments, a composition or formulation described herein comprises a viscosity enhancing agent that imparts on the composition a viscosity sufficient to provide increased residence on the esophagus while also allowing migration of the active agent(s) (solute or particles) when the composition is orally administered to an individual. In other words, in some embodiments, the viscosity is high enough to increase residence time of the composition on a gastrointestinal surface (e.g., a mucosal membrane or epithelium), but not so high as to prevent migration of the active agent(s) within the composition, e.g., toward the gastrointestinal surface (e.g., a mucosal membrane or epithelium). As used herein, delivery of a composition described herein to a gastrointestinal surface, mucosa, mucosal membrane, or epithelium are used interchangeably and refer to delivery of the composition to the surface tissue of the gastrointestinal tract, or of a specific site of the gastrointestinal tract.

In certain embodiments, a pharmaceutical composition described herein is a non-Newtonian fluid or a Newtonian fluid. In some embodiments, a pharmaceutical composition described herein is a non-Newtonian fluid. In specific embodiments, the non-Newtonian fluid is a plastic, pseudo-plastic or dilatant non-Newtonian fluid. In some specific embodiments, the non-Newtonian fluid is thixotropic. In certain embodiments, the non-Newtonian fluid composition thins with shear, and thickens upon the absence of shear. Thus, in some embodiments, provided herein is a fluid pharmaceutical composition that is suitable for easy pouring following mild or moderate agitation. Furthermore, in some embodiments, provided herein is a fluid pharmaceutical composition that while being suitable for easy pouring following mild or moderate agitation becomes viscous enough upon oral administration to allow the pharmaceutical composition to at least partially coat the esophagus and topically deliver a therapeutically effective amount of therapeutic agent to the esophagus. In some embodiments, the at least one additional excipient is selected from a non-Newtonian viscosity enhancing agent (i.e., an agent that provides a composition herein with a non-Newtonian character). Non-Newtonian viscosity enhancing agents include, by way of non-limiting example, acacia (e.g., used in about 5-10% w/w of a pharmaceutical composition described herein), alginic acid (e.g., about 0.5-20% w/w), carbomer, CaCMC, NaCMC, carrageenan (e.g., about 0.3-12% w/w), ceratonia (e.g., about 0.1-1% w/w), chitosin (e.g., about 0.5-2% w/w), colloidal silicon dioxide (e.g., about 2-10% w/w), ethylcellulose (e.g., about 5-25% w/w), gelatin, guar gum (e.g., about 1-2.5% w/w), HEC, hydroxyethylmethyl cellulose (e.g., about 1-5% w/w), hydroxypropyl cellulose (e.g., about 1-10% w/w), HPMC, magnesium aluminum silicate (e.g., about 2-10% w/w), one or more maltodextrin, methylcellulose (e.g., about 1-2% w/w), polyethylene glycol (e.g., about 45-60% w/w), povidone (e.g., about 10-15% w/w), saponite, sodium alginate (e.g., about 1-5% w/w), sucrose (e.g., about 50-70% w/w), tragacanth (e.g., about 0.1-2% w/w), xanthan gum (e.g., about 0.1-1% w/w), an combinations thereof.

In some embodiments, a pharmaceutical composition described herein is a Newtonian fluid. A Newtonian fluid can be described as a fluid whose viscosity is equal to the shear stress exerted by the fluid divided by the velocity gradient perpendicular to the direction of the shear. In certain embodiments, the one or more excipient that increases the interaction of the composition with a gastrointestinal surface is selected from a Newtonian viscosity enhancing agent (i.e., an agent that provides a composition herein with a Newtonian character). Newtonian viscosity enhancing agents include, by way of non-limiting example, glycerin (e.g., about 50-80% w/w), polydextrose (e.g., about 50-70% w/w), and combinations thereof.

In certain embodiments, following administration of a composition described herein to a gastrointestinal (e.g., esophageal) surface, at least 10%, 20%, 30%, 40%, 50%, 60%, 70%, 80%, 90% or 95% by weight of the therapeutic agent or composition administered resides upon, adheres to and/or is absorbed by the gastrointestinal (e.g., esophageal) surface after at least 0.25, 0.5, 0.75, 1, 1.5, 2, 3, 4, 5, 6, 7, 8, 9, 10, 15, 20, 25, 30, 35, 40, 45, 50, 55 or 60 minutes following application of the composition to the gastrointestinal (e.g., esophageal) surface. In specific embodiments, the gastrointestinal surface is or is adjacent to the gastrointestinal site afflicted by the gastrointestinal disorder (e.g., the site of gastrointestinal inflammation).

In certain embodiments, non-viscous compositions have a viscosity of about 0.2 cP to about 5 cP, about 1 cP or about the viscosity of water. In some embodiments, low-viscous compositions have a viscosity of less than 50 cP (e.g., about 1 cP, about 1 cP to about 40 cP, about 1 cP to about 30 cP, about 1 cP to about 20 cP, about 1 cP to about 10 cP, or the like). In certain embodiments, viscosities are measured at a shear rate of about 13.2 sec⁻¹ (e.g., with a gap between the spindle and sample chamber wall of about 6.0 mm or more). In some embodiments, a non-viscous or low-viscous composition described herein has an increased residence time upon a gastrointestinal (e.g., esophageal) surface as a result of the mucoadhesive character of the composition (e.g., the composition comprises a mucoadhesive agent that is non-viscous).

In some embodiments, a pharmaceutical composition described herein is sufficiently spreadable and/or has an appropriate flow characteristic on a gastrointestinal (e.g., esophageal) surface (e.g., mucosal or epithelial). In certain embodiments, the spreadability and/or flow characteristic of the composition is suitable so as to allow a pharmaceutical composition or a unit dose of a pharmaceutical composition described herein to spread across and/or flow upon the gastrointestinal surface site and at least partially coat the gastrointestinal surface site. In some embodiments, by at least partially coating the gastrointestinal surface, topical delivery of the therapeutic agent to the gastrointestinal site afflicted by the gastrointestinal disorder is achieved.

In certain embodiments, the pharmaceutical compositions provided herein are used to treat, prevent or alleviate gastrointestinal disorders or symptoms thereof including the esophagus, stomach and/or digestive tract. In specific embodiments, the pharmaceutical composition is in liquid form. Liquid forms include, by way of non-limiting example, solutions, suspensions, syrups, slurries, dispersions, colloids and the like. In specific embodiments, the liquid is a suspension. In some embodiments, a pharmaceutical composition described herein is in liquid, semi-solid or solid form. In specific embodiments, a pharmaceutical composition described herein is in semi-solid form, e.g., a gel, a gel matrix, a cream, a paste, or the like. In some embodiments, semi-solid forms comprise a liquid vehicle.

In some embodiments, provided herein are processes of adjusting the solubility of a lipophilic drug in a composition described herein comprising an aqueous medium by varying the concentration of one or more saccharide in the aqueous medium. In certain instances, the solubility of a lipophilic drug in an aqueous medium is increased by adding an acyclic oligosaccharide (e.g., maltodextrin or hyaluronic acid) to the aqueous medium. In some instance, the solubility of a lipophilic drug in an aqueous medium is decreased by adding a disaccharide or monosaccharide (e.g., dextrose) to the aqueous medium.

The methods and compositions of the present invention are used by individuals of any age. By “individual” is meant any animal, for example, a mammal, or, for example, a human, including, for example, patients in need of treatment. In some embodiments, the individual is a human adult. In other embodiments, the individual is a human child or infant. In certain embodiments, the human child or infant is less than 16 years old, less than 12 years old, less than 8 years old, less than 6 years old, less than 4 years old or less than 2 years old.

Formulations

While any of the pharmaceutical compositions, methods and kits described herein are typically used in therapy for human patients, in certain embodiments, they are used in veterinary medicine to treat similar or identical diseases. In some embodiments, the compositions are used, for example, to treat mammals, including, but not limited to, primates and domesticated mammals. In some embodiments, the compositions, methods and kits are used, for example, to treat herbivores. The compositions of the present invention include geometric and optical isomers.

Pharmaceutical compositions suitable for use in the present invention include compositions wherein the active ingredient or ingredients are contained in an effective amount to achieve its intended purpose. In certain embodiments, the pharmaceutical compositions disclosed herein comprise therapeutic agent in an amount sufficient to treat, prevent or alleviate disorders of the gastrointestinal tract (e.g., gastrointestinal inflammation), including the esophagus.

In certain embodiments, the exact dosage of therapeutic agent depends upon, by way of non-limiting example, the form in which the composition is administered, the subject to be treated, the age, body weight and/or height of the subject to be treated, and/or the preference and experience of the attending physician. Thus, in some embodiments, the dosage of therapeutic agent administered may vary from those disclosed herein. In certain embodiments, the optimal concentration of the therapeutic agent in the composition depends upon, by way of non-limiting example, the specific therapeutic agent used, the characteristics of the patient, and/or the nature of the inflammation for which the treatment is sought. In various embodiments, these factors are determined by those of skill in the medical and pharmaceutical arts in view of the present disclosure.

Generally, a therapeutically effective dose is desired. A therapeutically effective dose refers to the amount of the therapeutic agent that results in a degree of amelioration of symptoms and/or affliction relative to the status of such symptoms and/or affliction prior to treatment. The dosage forms and methods of applying dosage forms containing effective amounts are within the scope of the instant invention. In various embodiments, the amount of therapeutic agent used in a method or in a composition described herein is from about 1 μg/kg to about 10 mg/kg of body weight per day, for example about 1 μg/kg to about 1 mg/kg per day, about 1 μg/kg to about 400 μg/kg of body weight per day, about 5 to about 300 μg/kg per day, or about 5 to about 200 μg/kg per day.

In an illustrative embodiment, a dosage or amount (including a divided dose) of therapeutic agent is provided in a composition of sufficient volume to allow any of the compositions disclosed herein to reach the targeted and/or afflicted portion of the gastrointestinal tract, including, e.g., the esophagus, in an effective amount. In some embodiments, the effective amount of the composition delivered to the targeted or afflicted portion of the gastrointestinal tract (e.g., esophagus) is an amount sufficient to coat or at least partially coat the targeted or afflicted portion of the gastrointestinal tract (e.g., esophagus), and deliver the composition to the affected areas, including by way of example only, the lower esophagus, the esophageal-stomach juncture, the stomach and/or the duodenum. In certain embodiments, a composition described herein as a volume of, for example about 1-50 mL, or for example about 140 mL, or for example about 1-30 mL, or for example about 1-25 mL, or for example 1-20 mL, or for example about 5-25 mL, or for example about 10-20 mL, or for example about 10 mL, or for example, about 15 mL, or for example, about 20 mL, or for example about 1-15 mL, or for example about 1-10 mL, or for example about 2-8 mL, or for example about 3-7 mL, or for example, about 4-6 mL, or for example, about 5 mL, or for example about 6-14 mL, or for example about 8-12 mL, or for example, about 9-11 mL, or for example, about 10 mL. In more specific embodiments, about 0.01 mg to about 1 g, about 0.1 mg to about 0.5 g, about 0.1 mg to about 0.4 g, about 0.1 mg to about 0.3 g, about 0.1 mg to about 0.2 g, about 0.1 mg to about 100 mg, about 0.1 mg to about 80 mg, about 0.1 mg to about 50 mg, about 0.1 mg to about 40 mg, about 0.1 mg to about 30 mg, about 0.1 to about 25 mg, or about 1 mg to about 25 mg of therapeutic agent is formulated into a single or unit dose of a pharmaceutical composition described herein, the single or unit dose having a total volume of about 1-20 mL, about 10-20 mL, or for example about 10 mL, or for example, about 15 mL, or for example, about 20 mL, or for example about 1-15 mL, or for example about 1-10 mL, or for example about 2-8 mL, or for example about 3-7 mL, or for example, about 4-6 mL, or for example, about 5 mL, or for example about 6-14 mL, or for example about 8-12 mL, or for example, about 9-11 mL, or for example, about 10 mL.

As discussed herein, liquid encompasses slurries, solutions, suspensions, dispersions or any combination thereof, depending on the solubilities and amounts of the individual components and the vehicles and solvents used. In some embodiments, an appropriate palatable dosage is in a volume sufficient to coat or at least partially coat the esophagus, and in an illustrative embodiment, the volume is sufficient to coat or at least partially coat the esophagus and deliver the therapeutic agent to the affected areas (used interchangeably herein with afflicted areas), including by way of example only, the lower esophagus, the esophageal-stomach juncture, the stomach and/or the duodenum. The composition may be delivered, for example, four times a day, three times a day, twice a day, once a day, every other day, three times a week, twice a week, or once a week. The dosage may, for example, be divided into multiple doses throughout the day, or be provided, for example, in four, three, two, or one dose a day. In certain instances, more frequent administration (e.g., b.i.d. versus once a day) provides for a shorter overall therapy or a quicker onset of symptom resolution. In one illustrative example, the dose is provided once a day or at least once a day.

In certain embodiments, a dose or composition described herein is administered with food. In some embodiments, a dose or composition described herein is administered without food. In certain embodiments, a dose or composition described herein is administered in a fed or fasted state. In some embodiments, a dose or composition described herein is administered in the morning, in the afternoon, in the evening, at night, or a combination thereof. In some embodiments, the dose is administered at night. In another aspect, the dose is administered about 30 minutes prior to bed, with no food or water given after administration of the compositions herein. In yet another embodiment of the instant invention, the dose is administered prior to bedtime, wherein after administration of the composition, the patient or individual is in a substantially supine position for at least 30 minutes, at least 1 hour, at least 2 hours, at least 4 hours or at least 8 hours.

In some embodiments, provided herein are methods of treating, preventing, or alleviating inflammation or symptoms associated with inflammation of the gastrointestinal tract, e.g., the esophagus, comprising administering to an individual in need thereof a single unit dose of a pharmaceutical composition described herein from a multidose container. In specific embodiments, administering a single unit dose from a multi dose container comprises (1) shaking a multidose container, the multidose container comprising at least one unit dose of a pharmaceutical composition described herein; (2) pouring (or otherwise dispensing) a single unit dose from the multidose container into an administration device (e.g., a device suitable for administering to a human individual, such as a spoon, cup or syringe); and (3) administering the single unit dose to the individual in need thereof. In more specific embodiments, shaking of the multidose container occurs until the fluid therein has a viscosity suitable for pouring (e.g., easy pouring). In some specific embodiments, the process further comprises waiting after pouring the single unit dose and prior to administering the single unit dose to the individual in need thereof. In specific embodiments, the wait time is a time sufficient to allow the viscosity of composition to achieve a desired level, e.g., a viscosity to improve the coating capabilities of the composition. In some embodiments, the wait time is, e.g., about 3 seconds, or more; about 5 seconds, or more; about 10 seconds, or more; about 15 seconds, or more; about 20 seconds, or more; about 25 seconds, or more; about 30 seconds, or more; about 40 seconds, or more; about 45 seconds, or more; about 50 seconds, or more; or about 60 seconds, or more. In other specific embodiments, the composition is administered immediately following pouring the composition into the administration device. In some embodiments, the process comprises shaking the multidose container well.

In some embodiments, initial treatment continues, for example, for about 3 days to 2 weeks for an acute condition, or about 4 weeks to about 16 weeks for a chronic condition, or about 8 weeks to about 12 weeks for a chronic condition. In various embodiments, longer therapy is needed, such as, for example, therapy similar to chronic therapy for persistent asthma. In some aspects of the present invention, patients are, for example, be treated for up to 6 months, or up to one year. In certain aspects, maintenance treatments last up to or longer than one year. In some embodiments, patients are treated on a maintenance basis or on an as needed basis during a problematic episode, depending on the severity of the condition. In certain embodiments, patients are treated on a rotating treatment basis, where treatment is provided for a period of time and then the patient is taken off of the drug for a period before treatment resumes again. When off the drug, the patient may be given no treatment, treatment with another medication, or treatment with a reduced dosage. In certain embodiments, patients are given treatment with a higher dose of the composition until a desired reduced disease state is achieved, and then continued on a lower dose of the composition.

In some embodiments, the therapeutic agent is present in a pharmaceutical composition described herein in any effective amount. In some embodiments, an effective amount is an amount sufficient to treat or alleviate the disorder or to reduce symptoms of the disorder as compared to the level of affliction or symptoms of the disorder prior to administration of the effective amount. In certain embodiments, an effective amount is an amount sufficient to maintain a reduction in affliction or symptoms of the disorder and is achieved in any suitable manner including, but not limited to, by the administration of an effective amount sufficient to achieve such a reduction. In certain embodiments, the therapeutic agent is present in a pharmaceutical composition at a concentration of about 0.01 mg/mL to about 100 mg/mL, or about 0.01 mg/mL to about 50 mg/mL, or about 0.01 mg/mL to about 25 mg/mL of composition.

In certain embodiments, compositions described herein are useful and suitable for administration to children less than 10 years of age, e.g., 2-9. In some embodiments, compositions useful and suitable for administration to children less than 10 years of age (e.g., 2-9) comprise have a volume of about 7 mL. In some embodiments, compositions useful and suitable for administration to children or infants have a smaller volume than those administered to adults. In certain instances, the smaller volumes are useful for avoiding excessive systemic exposure by the children or infants to the therapeutic agent or agents present in the composition. In specific embodiments, compositions useful and suitable for administration to children less than 10 years of age (e.g., 2-9) comprise about 1 mg of corticosteroid (e.g., budesonide).

In some embodiments, compositions described herein are useful and suitable for administration to children of about 10 years of age to about 17 years of age. In some embodiments, compositions useful and suitable for administration to children 10 years of age to about 17 years of age comprise have a volume of about 7 mL or about 10 mL. In specific embodiments, compositions useful and suitable for administration to children of about 10 years of age to about 17 years of age comprise about 1 mg or about 2 mg of corticosteroid (e.g., budesonide). In further embodiments, compositions described herein are useful and suitable for administration to adults, e.g., adults of about 18 years of age to about 45 years of age. In some embodiments, compositions useful and suitable for administration to adults comprise have a volume of about 10 mL. In specific embodiments, compositions useful and suitable for administration to adults comprise about 1 mg or about 2 mg of corticosteroid (e.g., budesonide).

In some embodiments, the volume of a composition or dose of a composition described herein is an amount sufficient to substantially coat (e.g., at least 50%, at least 60%, at least 70%, at least 80%, at least 90%, at least 95%, at least 98% or at least 99% of) the length of the esophagus of an individual to whom the composition is administered. In certain embodiments, the volume of a composition or a dose of a composition described herein is about 0.05 mL/cm esophageal length to about 1 mL/cm esophageal length, about 0.1 mL/cm esophageal length to about 0.8 mL/cm esophageal length, about 0.2 mL/cm esophageal length to about 0.6 mL/cm esophageal length, or about 0.3 mL/cm esophageal length to about 0.5 nm/cm esophageal length, wherein the esophageal length is the esophageal length of the individual to whom the composition is administered. In some embodiments, the volume of a composition or dose of a composition described herein is based on the esophageal length of an individual (e.g., male, female, or both) that is in the 50^{th} percentile of height for their age. Therefore, in some embodiments, the volume of a composition or dose of a composition described herein is about 0.05 mL/cm esophageal length to about 1 mL/cm esophageal length, about 0.1 ml/cm esophageal length to about 0.8 mL/cm esophageal length, about 0.2 mL/cm esophageal length to about 0.6 mL/cm esophageal length, about 0.3 mL/cm esophageal length to about 0.5 mL/cm esophageal length, about 0.32 mL/cm esophageal length to about 0.41 ml/cm esophageal length, or about 0.3 mL/cm esophageal length to about 0.46 mL/cm esophageal length, wherein the esophageal length is the esophageal length of an individual having a height in the 50^{th} percentile for the age of the individual to whom the composition is administered. In certain instances, esophageal length is the actual esophageal length of the individual or is calculated based on the equation: esophageal length=1.048 (cm)+(0.167*height (cm)). In certain instances, for example, the 50^{th} percentile height (CDC 2000) for male children age 2 is 87 cm, age 3 is 95 cm, age 4 is 102 cm age 5 is 109 cm age 6 is 115 cm, age 7 is 122 cm, age 8 is 128 cm, age 9 is 134 cm, age 10 is 139 cm, age 11 is 144 cm, age 12 is 149 cm age 13 is 156 cm, age 14 is 164 cm, age 15 is 170 cm age 16 is 174 cm, age 17 is 175 cm, and age 18 is 176 cm.

Furthermore, in certain embodiments, the amount of a therapeutic agent (e.g., a corticosteroid such as budesonide) in a composition or a dose of a composition described herein is about 0.005 mg/cm esophageal length to about 0.3 mg/cm esophageal length, about 0.008 mg/cm esophageal length to about 0.2 mg/cm esophageal length, about 0.01 mg/cm esophageal length to about 0.15 mg/cm esophageal length, or about 0.015 mg/cm esophageal length to about 0.1 mg/cm esophageal length, wherein the esophageal length is the esophageal length of the individual to whom the composition is administered. In some embodiments, the volume of a composition or dose of a composition described herein is based on the esophageal length of an individual (e.g., male, female, or both) that is in the 50^{th} percentile of height for their age. Therefore, in some embodiments, the amount of a therapeutic agent (e.g., a corticosteroid such as budesonide) in a composition or dose of a composition described herein is about 0.005 mg/cm esophageal length to about 0.3 mg/cm esophageal length, about 0.008 mg/cm esophageal length to about 0.2 mg/cm esophageal length, about 0.01 mg/cm esophageal length to about 0.15 mg/cm esophageal length, or about 0.015 mg/cm esophageal length to about 0.1 mg/cm esophageal length, wherein the esophageal length is the esophageal length of an individual having a height in the 50^{th} percentile for the age of the individual to whom the composition is administered.

In some embodiments, any pharmaceutical composition or dose of a pharmaceutical composition described herein is provided or administered in a volume sufficient to provide a bolus when orally administered to an individual. In certain embodiments, the composition has a volume that does not systemically deliver excessive amounts of the active agent. In some embodiments, the pharmaceutical composition or dose is provided in a volume sufficient to provide a bolus when administered to an individual, wherein the size of the bolus at the distal end of the esophagus (e.g., the size of the bolus prior, e.g., immediately prior, to entering or passing the lower esophageal sphincter) is less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, less than 10% or less than 5% of size of the bolus that entered the esophagus (e.g., the size of the bolus after, e.g., immediately after, passing the upper esophageal sphincter). In some embodiments, the size of the bolus is determined as a measure of diameter or of volume. In certain embodiments, diameter of the sphincter can be determined using gamma scintigraphy techniques. In specific embodiments, the volume of the composition or dose is adjusted given the length and/or diameter of the esophagus of the individual to whom the composition or dose is administered.

In some embodiments, the concentration of a therapeutic agent (e.g., a corticosteroid such as budesonide) in a composition or a dose of a composition described herein is about 0.0001 mg/mL/cm esophageal length to about 10 mg/mL/cm esophageal length, about 0.0001 mg/mL/cm esophageal length to about 1 mg/mL/cm esophageal length, or about 0.001 mg/mL/cm esophageal length to about 0.5 mg/mL/cm esophageal length, wherein the esophageal length is as described herein.

In some embodiments, a composition described herein comprises a therapeutic agent, one or more excipient that increases the interaction of the composition with a gastrointestinal surface (e.g., an agent that enhances viscosity, mucoadhesive character, adsorption to a gastrointestinal surface, and/or absorption of an active through a gastrointestinal surface), optionally one or more binder, optionally one or more filler, optionally one or more lubricant, optionally one or more solvent (or vehicle), optionally one or more suspension agent, optionally one or more flavoring agent, optionally one or more coloring agent optionally one or more sweetener, optionally one or more preservative, optionally one or more antioxidant, optionally one or more buffering agent, optionally one or more humectant, optionally one or more chelating agent, and optionally one or more surfactant. In specific embodiments, the composition comprises a preservative. In further or alternative embodiments, the composition comprises a flavoring agent. In further or alternative embodiments, the liquid vehicle is an aqueous medium (e.g., water). In specific embodiments, particles (e.g., microparticles) comprising the therapeutic agent are suspended in the aqueous medium.

In some embodiments, the at least one additional excipient comprises or is HPMC. In specific embodiments, HPMC is present in the composition in an amount of about 0.01 mg/mL to about 500 mg/mL, about 0.1 mg/mL to about 200 mg/mL, about 1 mg/mL to about 100 mg/mL, about 30 mg/mL to about 70 mg/mL or about 5 mg/mL to about 50 mg/mL. In some embodiments, the at least one additional excipient comprises or is CMC. In specific embodiments, CMC is present in the composition in an amount of 0.01 mg/mL to about 500 mg/mL, about 0.1 mg/mL to about 200 mg/mL, about 1 mg/mL to about 100 mg/mL, about 30 mg/mL to about 70 mg/mL or about 5 mg/mL to about 50 mg/mL. In some embodiments, the at least one additional excipient comprises or is MCC. In specific embodiments, MCC is present in the composition in an amount of 0.01 mg/mL to about 500 mg/mL, about 0.1 mg/mL to about 200 mg/mL, about 1 mg/mL to about 100 mg/mL, about 30 mg/mL to about 70 mg/mL or about 5 mg/mL to about 50 mg/mL. In certain embodiments, the at least one additional excipient comprises or is carbomer. In specific embodiments, carbomer is present in the composition in an amount of 0.01 mg/mL to about 500 mg/mL, about 0.1 mg/mL to about 200 mg/mL, about 2 mg/mL to about 250 mg/mL, or about 5 mg/mL to about 100 mg/mL. In some embodiments, the at least one additional excipient comprises or is HEC. In specific embodiments, HEC is present in the composition in an amount of 0.01 mg/mL to about 500 mg/mL, about 0.1 mg/mL to about 200 mg/mL, about 1 mg/mL to about 100 mg/mL, about 30 mg/mL to about 70 mg/mL or about 5 mg/mL to about 50 mg/mL. In some embodiments, the at least one additional excipient comprises or is a combination of CMC and MCC (e.g., Avicel® RC-591). In specific embodiments, the CMC/MCC combination (e.g., Avicel® RC-591) is present in the composition in an amount of 0.01 mg/mL to about 500 mg/mL, about 0.1 mg/mL to about 200 mg/mL, about 1 mg/mL to about 150 mg/mL, 1 mg/mL to about 75 mg/mL, or about 5 mg/mL to about 40 mg/mL. In certain embodiments, the CMC/MCC mixed weight ratio is between about 1/99 and about 99/1, about 20/80 and about 5/95, or about 15/85 and about 10/90. In a specific embodiment, the CMC is NaCMC and the CMC/MCC mixed weight ratio is about 11/89.

In certain embodiments, dextrose is present in the composition in an amount of 0.01 mg/mL to about 2 g/mL about 0.1 mg/mL to about 1 g/mL, about 10 mg/mL to about 1 g/mL. In some embodiments, maltodextrin is present in the composition in an amount of 0.01 mg/mL to about 2 g/mL, about 0.1 mg/mL to about 1.5 g/mL, about 10 mg/mL to about 1.5 g/mL. In certain embodiments, edetate is present in a composition in an amount of about 0.02 mg/mL to about 25 mg/mL, about 0.02 mg/mL to about 5 mg/mL, about 0.02 mg/mL to about 2 mg/mL, or about 0.05 mg/mL to about 2 mg/mL. In some embodiments, citrate is present in the composition in an amount of about 0.1 mg/mL to about 50 mg/mL, or about 0.2 mg/mL to about 30 mg/mL. In certain embodiments, polysorbate 80 is present in the composition in an amount of about 0.01 mg/mL to about 10 mg/mL, about 0.05 mg/mL to about 1 mg/mL, or about 0.05 to about 0.5 mg/mL.

In certain embodiments, amounts of component per mL refers to the amount of component in relation to the amount of total (or q.s.) volume of the composition as a whole, rather than the volume of any liquid component, such as water, alone.

In some embodiments, initial treatment continues, for example, for about 3 days to 2 weeks for an acute condition, or about 4 weeks to about 16 weeks for a chronic condition, or about 8 weeks to about 12 weeks for a chronic condition. In various embodiments, longer therapy is needed, such as, for example, therapy similar to chronic therapy for persistent asthma. In some aspects of the present invention, patients are, for example, be treated for up to 6 months, or up to one year. In certain aspects, maintenance treatments last up to or longer than one year. In some embodiments, patients are treated on a maintenance basis or on an as needed basis during a problematic episode, depending on the severity of the condition. In certain embodiments, patients are treated on a rotating treatment basis, where treatment is provided for a period of time and then the patient is taken off of the drug for a period before treatment resumes again. When off the drug, the patient may be given no treatment, treatment with another medication, dietary therapy, or treatment with a reduced dosage. In certain embodiments, patients are given treatment with a higher dose of the composition until a desired reduced disease state is achieved, and then continued on a lower dose of the composition. In certain embodiments, a patient combines treatment with a composition described herein with a treatment with another medication, and/or dietary therapy. In certain embodiments, patients are given treatment with a higher dose of the composition until a desired reduced disease state is achieved, and then continued on a lower dose of the composition.

In certain embodiments, the compositions described herein further comprise excipients and/or auxiliaries suitable for enabling the compositions to be formulated tablets, pills, dragees, capsules, liquids, soft chews, creams, pastes, chewable tablets, gels or gel matrices, syrups, slurries, suspensions, gums, lozenges, and the like, for oral ingestion by a patient to be treated. In certain instances, oral formulations (e.g., suspensions, creams or gel matrices) are formulated such that upon oral administration, an interface layer between the oral formulation (e.g., suspension, cream or gel matrix) and a gastrointestinal surface (e.g., gastrointestinal mucosa and/or gastrointestinal epithelium) is formed. In some instances, an oral formulation (e.g., suspensions, creams or gel matrices) in contact with a gastrointestinal surface delivers a therapeutic agent to or through the gastrointestinal surface via the interface layer and as the oral formulations (e.g., suspensions, creams or gel matrices) near the interface layer is depleted of therapeutic agent, a concentration gradient results. In certain instances, portions of the oral formulations (e.g., suspensions, creams or gel matrices) with high concentrations of therapeutic agent relative to the portions of the oral formulations (e.g., suspensions, creams or gel matrices) proximate to the interface layer replenishes therapeutic agent in the portion of the oral formulations (e.g., suspensions, creams or gel matrices) proximate to the interface layer. In certain instances, upon oral administration of an oral formulation described herein to an individual, an interface layer is formed between a gastrointestinal surface and a mixture of the oral formulation (e.g., chewable tablet) and saliva of the individual. Discussion of interactions between pharmaceutical compositions described herein with a gastrointestinal surface includes the disclosure of the interaction achieved between such saliva mixtures and the gastrointestinal surface.

In certain embodiments, the pharmaceutical compositions described herein optionally comprise water. In certain embodiments, the pharmaceutical compositions described herein comprise water as a vehicle. In some embodiments, the vehicle is a combination of water and alcohol. In other embodiments, the vehicle is a non-aqueous liquid vehicle. In certain instances, a liquid vehicle dissolves or partially dissolves the therapeutic agent.

Diseases

In some embodiments, provided herein are methods of treating, preventing, or alleviating disorders or symptoms associated with the gastrointestinal tract, e.g., the esophagus. In certain embodiments, provided herein are methods of treating diseases or conditions of the gastrointestinal tract, e.g., the esophagus, by administering a composition described herein. In specific embodiments, administration of the composition described herein treats, prevents, or alleviates the gastrointestinal disorder (including symptoms of a disease or disorder that present in the gastrointestinal tract). Disorders of the gastrointestinal tract include, by way of non-limiting example, gastrointestinal inflammation, gastrointestinal cancer, gastrointestinal infection (e.g., bacterial or fungal), gastrointestinal motility dysfunction (e.g., esophageal dysmotility), or gastrointestinal lesions, wounds or contusions. More specifically, disorders of the gastrointestinal tract include, by way of non-limiting example, esophageal inflammation, esophageal cancer, esophageal infection (e.g., bacterial or fungal), esophageal motility dysfunction, or esophageal lesions, wounds or contusions. Diseases or conditions of the gastrointestinal tract include, by way of non-limiting example, any chronic inflammatory or malignant state that involves the gastrointestinal tract (e.g., the esophagus, stomach and/or digestive tract) and responds to steroid therapy. The methods of the present invention are useful, for example, for treating, preventing and alleviating the inflammation associated with or symptoms of eosinophilic esophagitis, intermediate esophagitis (IE), epithelial hyperplasia, basal cell hyperplasia, elongated papillae, dilated vessels in papillae, fungal esophagitis, viral esophagitis, bacterial esophagitis, corrosive esophagitis, radiation esophagitis, chemotherapy esophagitis, eosinophilic gastric outlet obstruction and related inflammation, graft vs. host disease, a skin disease with esophageal involvement, bullous pemphigoid, pemphigus vulgaris, epidermolysis bollosa, Stevens-Johnson syndrome, Behçet's disease, sarcoidosis, idiopathic esophagitis, eosinophilic gastritis, Ménétrier's disease, parasitic gastritis, lymphocytic esophagitis, inflammatory bowel disease-associated esophagitis, parasitic gastritis, lymphocytic esophagitis, inflammatory bowel disease-associated esophagitis, tracheoesophageal fistulae (TEF), inflammatory bowel diseases involving the esophagus, reflux esophagitis, Crohn's disease, proximal gastrointestinal pathology (e.g., in individuals suffering from hypofunctioning gallbladder), eosinophilic gastrointestinal inflammation, celiac disease, eosinophilic duodenitis, duodenal eosinophilia, functional dyspepsia, acute esophageal inflammation secondary to caustic/irritant ingestion, persistent/recurrent esophageal strictures secondary to caustic/irritant, conditions due to ingestion, systemic diseases, congenital diseases, post-surgery inflammation, and gastro enteritis. The methods of the present invention are also useful, for example, for treating, preventing and alleviating inflammation associated with or symptoms of reflux esophagitis, gastroesophageal reflux disease (GERD), nonerosive reflux disease (NERD), Barrett's Esophagus, and/or erosive esophagitis. The methods of the present invention are also useful, for example, for treating, preventing and alleviating, by way of non-limiting example, celiac disease, drug allergy, connective tissue diseases, graft-vs-host disease, oral chronic graft-versus-host disease, radiation injury, chemical injury, oral/esophageal mucositis, oral/esophageal mucositis in cancer patients, and pharyngitis.

In certain embodiments, provided herein is a method of treating gastrointestinal inflammation (e.g., inflammation of the esophagus) in an individual by administering to an individual in need thereof a composition described herein, wherein the therapeutic agent is amount of a histamine (e.g., H1, H2, and/or H3) receptor ligand, a transient lower esophageal sphincter relaxation (TLESR)-reducing agent, a prokinetic serotonergic agent, a potassium-competitive acid blocker (P-CAB), a mucosal protectant, an anti-gastrin agent, a leukotriene antagonist, a mast cell inhibitor, a mast cell stabilizer, an immunomodulator, a biologic, an anti-asthmatic agent, a non-steroidal anti-inflammatory drug (NSAID), corticosteroid, mGluR₅ antagonists, acetylcholine modulator, 5HT₄ receptor agonist, 5HT₃ receptor antagonist, 5HT₁ receptor antagonist, an antibiotic, an antiseptic, an anesthetic, or a combination thereof. In specific embodiments, gastrointestinal inflammation treated according to the methods described herein include, by way of non-limiting example, eosinophilic esophagitis, intermediate esophagitis (IE), epithelial hyperplasia, basal cell hyperplasia, elongated papillae, dilated vessels in papillae, fungal esophagitis, viral esophagitis, bacterial esophagitis, corrosive esophagitis, radiation esophagitis, chemotherapy esophagitis, graft vs. host disease, a skin disease with esophageal involvement, bullous pemphigoid, pemphigus vulgaris, epidermolysis bollosa, Stevens-Johnson syndrome, Behçet's disease, sarcoidosis, idiopathic esophagitis, eosinophilic gastritis, Ménétrier's disease, parasitic gastritis, lymphocytic esophagitis, inflammatory bowel disease-associated esophagitis, parasitic gastritis, lymphocytic esophagitis, inflammatory bowel disease-associated esophagitis, tracheoesophageal fistulae (TEF), inflammatory bowel diseases involving the esophagus, Crohn's disease, proximal gastrointestinal pathology (e.g., in individuals suffering from hypofunctioning gallbladder), eosinophilic gastrointestinal inflammation, celiac disease, eosinophilic duodenitis, duodenal eosinophilia, functional dyspepsia, reflux esophagitis, acute esophageal inflammation secondary to caustic/irritant ingestion, persistent/recurrent esophageal strictures secondary to caustic/irritant, conditions due to ingestion, systemic diseases, congenital diseases, post-surgery inflammation, gastro enteritis, gastroesophageal reflux disease (GERD), nonerosive reflux disease (NERD), Barrett's Esophagus, and/or erosive esophagitis.

In some embodiments, provided herein is a method of treating cancer of the gastrointestinal tract (e.g., esophageal cancer) in an individual by administering to an individual in need thereof a composition described herein, wherein the therapeutic agent is a chemotherapeutic agent.

In certain embodiments, provided herein is a method of treating gastrointestinal (e.g., esophageal) motility dysfunction in an individual by administering to an individual in need thereof a composition described herein, wherein the therapeutic agent is a pro-motility agent, anti-motility agent, or a combination thereof.

In some embodiments, provided herein is a method of treating gastrointestinal (e.g., esophageal) infection in an individual by administering to an individual in need thereof a composition described herein, wherein the therapeutic agent is an antibiotic or antimicrobial agent. In specific embodiments, the antimicrobial agent is an anti-bacterial agent or an anti-fungal agent. In further or alternative embodiments, the infection is a bacterial or fungal infection.

In certain embodiments, provided herein is a method of treating eosinophilic esophagitis in an individual by administering to an individual in need thereof a composition described herein, wherein the therapeutic agent is, by way of non-limiting example, a corticosteroid, a leukotriene antagonist, a mast cell stabilizer/inhibitor, an immunomodulator, a biologic, or the like, or combinations thereof.

It will be appreciated that reference herein to treatment extends to prophylaxis as well as the treatment of inflammation or other disorders.

In certain embodiments, provided herein is a method of treating, preventing or alleviating disorders of the gastrointestinal tract, including, by way of non-limiting example, the esophagus, stomach and/or digestive tract, in an individual comprising orally administering to said individual any of the compositions described herein. In certain embodiments, the oral dosage form comprises a liquid vehicle and is formulated as, e.g., a slurry, suspension, syrup, dispersion, solution, or the like.

In some embodiments, the inflammation treated by the methods and compositions described herein is associated with mast cell inflammation, eosinophilic inflammation and/or neutrophilic inflammation. In some embodiments, individuals (e.g., patients) to be treated with compositions described herein include those that have been diagnosed with eosinophilic esophagitis, intermediate esophagitis (IE), epithelial hyperplasia, basal cell hyperplasia, elongated papillae, dilated vessels in papillae, fungal esophagitis, viral esophagitis, bacterial esophagitis, corrosive esophagitis, radiation esophagitis, chemotherapy esophagitis, graft vs. host disease, a skin disease with esophageal involvement, bullous pemphigoid, pemphigus vulgaris, epidermolysis bollosa, Stevens-Johnson syndrome, Behçet's disease, sarcoidosis, idiopathic esophagitis, eosinophilic gastritis, Ménétrier's disease, parasitic gastritis, lymphocytic esophagitis, inflammatory bowel disease-associated esophagitis, parasitic gastritis, lymphocytic esophagitis, inflammatory bowel disease-associated esophagitis, tracheoesophageal fistulae (TEF), an inflammatory bowel disease involving the esophagus, reflux esophagitis, Crohn's disease, proximal gastrointestinal pathology (e.g., in individuals suffering from hypofunctioning gallbladder), eosinophilic gastrointestinal inflammation, celiac disease, eosinophilic duodenitis, duodenal eosinophilia, functional dyspepsia, esophageal inflammation secondary to caustic/irritant ingestion, persistent/recurrent esophageal strictures of any cause and including caustic/irritant ingestion, pill-induced esophagitis, systemic diseases, congenital diseases, post-surgery inflammation, or gastro enteritis. In one non-limiting example, the patient has eosinophilic esophagitis. In some embodiments, individuals (e.g., patients) to be treated with the compositions described herein include those that have been diagnosed with gastroesophageal reflux disease (GERD), nonerosive reflux disease (NERD), Barrett's Esophagus, and/or erosive esophagitis. In some embodiments, individuals to be treated with the compositions described herein suffer from, by way of non-limiting example, gastrointestinal inflammation, gastrointestinal cancer, gastrointestinal infection (e.g., bacterial or fungal), gastrointestinal motility dysfunction, gastrointestinal lesions, wounds or contusions, celiac disease, drug allergy, connective tissue diseases, graft-vs-host disease, oral chronic graft-versus-host disease, radiation injury, chemical injury, oral/esophageal mucositis, oral/esophageal mucositis in cancer patients, and pharyngitis. In some embodiments, the patient is an adult. In other embodiments, the patient is a child or infant. In various aspects, a patient is a child or infant less than 16 years old, less than 12 years old, less than 8 years old, less than 6 years old, less than 4 years old or less than 2 years old.

In some embodiments, a composition is in a unit dose formulation for oral administration of a patient. In some embodiments, a unit dose of the therapeutic agent is administered from a metered dose device. In some embodiments, the metered dose device delivers a metered unit dose of a composition described herein to the mouth or throat of an individual in need thereof. In certain embodiments, the metered dose device is a metered inhaler, which is utilized to administer a metered unit dose to the mouth or throat of an individual (the individual swallows rather than inhales the metered unit dose). In certain embodiments, a metered dose device dispenses a metered unit dose of a composition described herein into a receptacle (e.g., a cup), which is then utilized to orally administer the metered unit dose to the mouth or throat.

In some embodiments, provided herein is a multiple unit container comprising about 2 to about 180, about 10 to about 60, about 14, or about 30 unit doses of any pharmaceutical composition described herein. In more specific embodiments, each dose comprises about 1 mL to about 25 mL, about 1 mL to about 20 mL, about 7 mL to about 25 mL, about 10 to about 20 mL, about 15 mL, about 20 mL, about 3 to about 7 mL, about 5 mL, about 8 mL to about 12 mL, or about 10 mL. In still more specific embodiments, each dose comprises about 0.1 to about 20 mg, about 0.1 to about 10 mg, about 0.1 to about 7.5 mg, about 0.1 to about 5 mg, about 0.3 to about 4 mg, about 0.25 to about 2.5 mg, about 0.3 mg to about 2 mg, about 0.5 mg to about 1 mg, about 0.7 mg to about 1.5 mg, about 0.375 mg, about 0.75 mg, about 1 mg, about 1.25 mg, about 1.5 mg or about 2 mg of corticosteroid. In certain embodiments, provided herein is a multiple unit container comprising about 10 mL to about 1500 mL, about 50 mL to about 600 mL, about 150 mL, about 300 mL, about 600 mL, or about 1,200 mL of any pharmaceutical composition described herein. In specific embodiments, the multidose container comprises about 330 mL or about 55 mL of a composition described herein. In some embodiments, a kit provided herein comprises any multidose container as described herein, a pharmaceutical composition as described herein (e.g., in a volume described), and a delivery or metered device (e.g., a syringe, a cup, a spoon, or the like). In specific embodiments, the delivery device is incorporated into the container (e.g., an nebulizer, a aerosolizer, a pump, or the like). In certain embodiments, the pharmaceutical composition contained within any of the multiple unit containers described herein is physically and chemically stable.

In some embodiments, provided herein is a process of diagnosing an individual with gastrointestinal a gastrointestinal disorder by (i) detecting and/or measuring symptoms of the disorder in an individual prior to administering to the individual a composition described herein; (ii) administering to the individual a composition described herein; (iii) detecting and/or measuring symptoms of the individual following administration of the composition; and (iv) comparing the symptoms measured or detected prior to and following administration of a composition described herein. If the symptoms exhibited by the individual are reduced (e.g., by a statistically significant or clinically relevant amount), a positive diagnosis occurs. In specific embodiments, the process of diagnosing an individual with gastrointestinal inflammation is diagnosing an individual with eosinophilic esophagitis.

The entirety of each patent, patent application, publication and document referenced herein is hereby incorporated by reference. Citation of the above patents, patent applications, publications and documents is not an admission that any of the foregoing is pertinent prior art, nor does it constitute any admission as to the contents or date of these publications or documents.

Unless defined otherwise, all technical and scientific terms used herein have the same meanings as commonly understood by one of ordinary skill in the art to which this invention belongs. Although any methods and systems similar or equivalent to those described herein can be used in the practice or testing of the present invention, the methods, devices, and materials are now described. All publications mentioned herein are incorporated herein by reference for the purpose of describing and disclosing the processes, systems, and methodologies which are reported in the publications which might be used in connection with the invention. Nothing herein is to be construed as an admission that the invention is not entitled to antedate such disclosure by virtue of prior invention.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any elements) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention.

While certain embodiments have been shown and described herein, it will be apparent to those skilled in the art that such embodiments are provided by way of example only. Numerous variations, changes, and substitutions will now occur to those skilled in the art and are considered to be within the scope of the disclosure herein. It should be understood that various alternatives to the embodiments of the invention described herein may be employed in practicing the invention. It is intended that the following claims define the scope of the invention and that methods and structures within the scope of these claims and their equivalents be covered thereby.

### EXAMPLES

### Example 1

### Formulation #1

An exemplary composition described herein is prepared by combining the following:

| Ingredient | Amount |
|---|---|
| Therapeutic Agent | 1 mg to 500 mg |
| Avicel (RC-591) | 0.5 g to 4 g |
| Disodium Edetate | 5 mg to 200 mg |
| Citric Acid | 10 mg to 1 g |
| Sodium Citrate | 10 mg to 2 g |
| Tween 80 | 5 mg to 100 mg |
| Flavoring Agent | optional |
| Sweetener | optional |
| Preservative | optional |
| Water | q.s. to 100 mL |

The composition is divided into a unit dose of about 1 mL to about 20 mL (e.g., about 5 mL, or about 10 mL) and administered orally to an individual to treat, prevent or alleviate a disorder of the gastrointestinal tract (e.g., the esophagus).

### Example 2

### Formulation #2

An exemplary composition described herein is prepared by combining the following:

| Ingredient | Amount |
|---|---|
| Therapeutic Agent | 1 mg to 500 mg |
| CMC | 0.5 g to 3 g |
| Disodium Edetate | 5 mg to 200 mg |
| Citric Acid | 10 mg to 1 g |
| Sodium Citrate | 10 mg to 2 g |
| Tween 80 | 5 mg to 100 mg |
| Flavoring Agent | optional |
| Sweetener | optional |
| Preservative | optional |
| Water | q.s. to 100 mL |

The composition is divided into a unit dose of about 1 mL to about 20 mL (e.g., about 5 mL, or about 10 mL) and administered orally to an individual to treat, prevent or alleviate a disorder of the gastrointestinal tract (e.g., the esophagus).

### Example 3

### Formulation #3

An exemplary composition described herein is prepared by combining the following:

| Ingredient | Amount |
|---|---|
| Therapeutic Agent | 1 mg to 500 mg |
| Carbomer | 0.5 g to 10 g |
| Disodium Edetate | 5 mg to 200 mg |
| Citric Acid | 10 mg to 1 g |
| Sodium Citrate | 10 mg to 2 g |
| Tween 80 | 5 mg to 100 mg |
| Flavoring Agent | optional |
| Sweetener | optional |
| Preservative | optional |
| Water | q.s. to 100 mL |

The composition is divided into a unit dose of about 1 mL to about 20 mL (e.g., about 5 mL, or about 10 mL) and administered orally to an individual to treat, prevent or alleviate a disorder of the gastrointestinal tract (e.g., the esophagus).

### Example 4

### Formulation #4

An exemplary composition described herein is prepared by combining the following:

| Ingredient | Amount |
|---|---|
| Therapeutic Agent | 1 mg to 500 mg |
| HPMC | 0.5 g to 3 g |
| Disodium Edetate | 5 mg to 200 mg |
| Citric Acid | 10 mg to 1 g |
| Sodium Citrate | 10 mg to 2 g |
| Tween 80 | 5 mg to 100 mg |
| Flavoring Agent | optional |
| Sweetener | optional |
| Preservative | optional |
| Water | q.s. to 100 mL |

The composition is divided into a unit dose of about 1 mL to about 20 mL (e.g., about 5 mL, or about 10 mL) and administered orally to an individual to treat, prevent or alleviate a disorder of the gastrointestinal tract (e.g., the esophagus).

### Example 5

### Formulation #5

An exemplary composition described herein is prepared by combining the following:

| Ingredient | Amount |
|---|---|
| Therapeutic Agent | 1 mg to 500 mg |
| MCC | 0.5 g to 3 g |
| Disodium Edetate | 5 mg to 200 mg |
| Citric Acid | 10 mg to 1 g |
| Sodium Citrate | 10 mg to 2 g |
| Tween 80 | 5 mg to 100 mg |
| Flavoring Agent | optional |
| Sweetener | optional |
| Preservative | optional |
| Water | q.s. to 100 mL |

The composition is divided into a unit dose of about 1 mL to about 20 mL (e.g., about 5 mL, or about 10 mL) and administered orally to an individual to treat, prevent or alleviate a disorder of the gastrointestinal tract (e.g., the esophagus).

### Example 6

### Formulation #6

An exemplary composition described herein is prepared by combining the following:

| Ingredient | Amount |
|---|---|
| Therapeutic Agent | 1 mg to 500 mg |
| Dextrose | 1 g to 100 g |
| Disodium Edetate | 5 mg to 200 mg |
| Citric Acid | 10 mg to 1 g |
| Sodium Citrate | 10 mg to 2 g |
| Tween 80 | 5 mg to 100 mg |
| Flavoring Agent | optional |
| Sweetener | optional |
| Preservative | optional |
| Water | q.s. to 100 mL |

The composition is divided into a unit dose of about 1 mL to about 20 mL (e.g., about 5 mL, or about 10 mL) and adminstered orally to an individual to treat, prevent or alleviate a disorder of the gastrointestinal tract (e.g., the esophagus).

### Example 7

### Formulation #7

An exemplary composition described herein is prepared by combining the following:

| Ingredient | Amount |
|---|---|
| Therapeutic Agent | 1 mg to 500 mg |
| Maltodextrin | 1 g to 100 g |
| Disodium Edetate | 5 mg to 200 mg |
| Citric Acid | 10 mg to 1 g |
| Sodium Citrate | 10 mg to 2 g |
| Tween 80 | 5 mg to 100 mg |
| Flavoring Agent | optional |
| Sweetener | optional |
| Preservative | optional |
| Water | q.s. to 100 mL |

The composition is divided into a unit dose of about 1 mL to about 20 mL (e.g., about 5 mL, or about 10 mL) and administered orally to an individual to treat, prevent or alleviate a disorder of the gastrointestinal tract (e.g., the esophagus).

### Example 8

### Formulation #8

An exemplary composition described herein is prepared by combining the following:

| Ingredient | Amount |
|---|---|
| Therapeutic Agent | 1 mg to 500 mg |
| Dextrose | 1 g to 100 g |
| Maltodextrin | 1 g to 100 g |
| Disodium Edetate | 5 mg to 200 mg |
| Citric Acid | 10 mg to 1 g |
| Sodium Citrate | 10 mg to 2 g |
| Tween 80 | 5 mg to 100 mg |
| Flavoring Agent | optional |
| Sweetener | optional |
| Preservative | optional |
| Water | q.s. to 100 mL |

The composition is divided into a unit dose of about 1 mL to about 20 mL (e.g., about 5 mL, or about 10 mL) and administered orally to an individual to treat, prevent or alleviate a disorder of the gastrointestinal tract (e.g., the esophagus).

### Example 9

### Formulation #9

An exemplary composition described herein is prepared by combining the following:

| Ingredient | Amount |
|---|---|
| Therapeutic Agent | 1 mg to 500 mg |
| HEC | 0.5 g to 5 g |
| Disodium Edetate | 5 mg to 200 mg |
| Citric Acid | 10 mg to 1 g |
| Sodium Citrate | 10 mg to 2 g |
| Tween 80 | 5 mg to 100 mg |
| Flavoring Agent | optional |
| Sweetener | optional |
| Preservative | optional |
| Water | q.s. to 100 mL |

The composition is divided into a unit dose of about 1 mL to about 20 mL (e.g., about 5 mL, or about 10 mL) and administered orally to an individual to treat, prevent or alleviate a disorder of the gastrointestinal tract (e.g., the esophagus).

### Example 10

### Budesonide Formulation

An exemplary composition described herein is prepared by combining the following:

| Ingredient | Amount | |
|---|---|---|
| Budesonide | 1 | mg |
| Avicel | 100 | mg |
| Dextrose | 0.5 | g |
| Maltodextrin (M150) | 1.3 | g |
| EDTA | 2.5 | mg |
| Tween 80 | 0.5 | mg |
| Cherry Flavor | 25 | mg |
| Glycerin | 250 | mg |
| AceK | 37.5 | mg |
| Magnasweet | 25 | mg |
| Sodium Benzoate | 10 | mg |
| Potassium Sorbate | 10 | mg |
| Aqueous Citric Acid Buffer | q.s. to 5 | mL |

The composition is administered orally to an individual to treat, prevent or alleviate inflammation or symptoms of inflammation of the gastrointestinal tract (e.g., the esophagus).

### Example 11

### Budesonide Formulation

An exemplary composition described herein is prepared by combining the following:

| Ingredient | Amount | |
|---|---|---|
| Budesonide | 1 | mg |
| Avicel | 200 | mg |
| Dextrose | 1 | g |
| Maltodextrin (M150) | 2.6 | g |
| EDTA | 5 | mg |
| Tween 80 | 1 | mg |
| Cherry Flavor | 50 | mg |
| Glycerin | 500 | mg |
| AceK | 75 | mg |
| Magnasweet | 50 | mg |
| Sodium Benzoate | 10 | mg |
| Potassium Sorbate | 10 | mg |
| Aqueous Citric Acid Buffer | q.s. to 10 | mL |

The composition is administered orally to an individual to treat, prevent or alleviate inflammation or symptoms of inflammation of the gastrointestinal tract (e.g., the esophagus).

### Example 12

In certain instances, the formulations described in the examples set forth herein are scaled to an amount sufficient to provide any desired amount, e.g., about 150 mL of total composition volume, either by scaling the up a composition described in the examples herein, e.g., to about 150 mL, or by scaling to a larger volume and dividing the scaled composition into smaller portions, e.g., portions comprising about 150 mL. The portions, e.g., those comprising about 150 mL, may then be placed into a multi dose container. A plurality of doses may then be dispensed and administered to an individual to treat, prevent or alleviate disorders of the gastrointestinal tract (e.g., the esophagus).

What is claimed is:

1. An oral pharmaceutical composition comprising:
a. a therapeutically effective amount of one or more therapeutic agent;
b. one or more excipient that increases the interaction of the composition with a gastrointestinal surface;
c. optionally, one or more sweetener, one or more flavoring agent, or a combination thereof; and
d. one or more pharmaceutically acceptable carrier, vehicle or a combination thereof;
wherein the oral pharmaceutical composition is physically and chemically stable; and wherein the oral pharmaceutical composition is topically active.

2. The pharmaceutical composition of claim 1, wherein the therapeutic agent is selected from a histamine antagonist, a transient lower esophageal sphincter relaxation (TLESR) reducing agent, a promotility agent, a prokinetic serotonergic agent, a potassium-competitive acid blocker (P-CAB), a mucosal protectant, an anti-gastrin agent, an anti-inflammatory agent, an antibacterial agent, an antifungal agent, a wound healing agent, a chemotherapeutic agent, a monoclonal antibody, an anti-emetic agent, erythropoietin, a synthetic erythropoietin, or combinations thereof.

3. The pharmaceutical composition of claim 1, wherein the composition remains substantially uniform after storage in ambient conditions for at least one month.

4. The pharmaceutical composition claim 1, wherein the composition is a solid, semi-solid, gel, cream, ointment, solution, dispersion, suspension, or paste.

5. The pharmaceutical composition claim 1, wherein the composition regains substantial uniformity upon mild or moderate agitation, swirling, gentle swirling or shaking.

6. The pharmaceutical composition of claim 1, wherein the pharmaceutical composition comprises a plurality of doses and the plurality of doses are in a multiple-unit container.

7. The pharmaceutical composition of claim 6, wherein each dose from the container of the formulation is substantially uniform with regard to each other.

8. The pharmaceutical composition of claim 6, wherein the first and final dose from the container are substantially uniform.

9. The pharmaceutical composition of claim 1, wherein the pharmaceutical composition comprises a plurality of particles comprising the therapeutic agent; wherein the vehicle is a liquid vehicle; and wherein the plurality of particles of the therapeutic agent are suspended in the liquid vehicle.

10. The pharmaceutical composition of claim 9, wherein the therapeutic agent is readily dispersed throughout the composition upon mild or moderate agitation.

11. The pharmaceutical composition of claim 9, wherein the therapeutic agent is readily suspended the composition upon mild or moderate agitation.

12. The pharmaceutical composition of claim 9, wherein the therapeutic agent is readily re-suspended in the composition upon mild or moderate agitation after storage in ambient conditions for one month.

13. The pharmaceutical composition of claim 1, wherein the pharmaceutical composition is a non-Newtonian fluid.

14. The pharmaceutical composition of claim 13, wherein the non-Newtonian fluid is thixotropic.

15. The pharmaceutical composition of claim 1, wherein the one or more excipient comprises one or more maltodextrin, dextrose, hydroxyethylcellulose (HEC), hydroxypropylmethyl-cellulose (HPMC), carboxymethyl-cellulose (CMC), microcrystalline cellulose (MCC), carbomer or combinations thereof.

16. The pharmaceutical composition of claim 1, wherein the therapeutic agent is topically active.

17. The pharmaceutical composition of claim 1, wherein the therapeutic agent is a microparticle.

18. The pharmaceutical composition of claim 1, wherein the therapeutic agent is a nanoparticle.

19. The pharmaceutical composition of claim 1, wherein the therapeutic agent is substantially dissolved in the vehicle or carrier.

20. The pharmaceutical composition of claim 1, wherein the vehicle is a liquid vehicle that comprises an aqueous medium.

21. The pharmaceutical composition of claim 1, wherein the one or more excipient comprises CMC, and wherein the CMC is present in an amount of about 5 mg/mL to about 30 mg/mL.

22. The pharmaceutical composition of claim 1, wherein the one or more excipient comprises carbomer, and wherein the carbomer is present in an amount of about 5 mg/mL to about 100 mg/mL.

23. The pharmaceutical composition of claim 1, wherein the one or more excipient comprises HPMC, and wherein the HPMC is present in an amount of about 5 mg/mL to about 30 mg/mL.

24. The pharmaceutical composition of claim 1, wherein the one or more excipient comprises MCC, and wherein the MCC is present in an amount of about 5 mg/mL to about 30 mg/mL.

25. The pharmaceutical composition of claim 1, wherein the one or more excipient comprises a combination of CMC and MCC, wherein the CMC-MCC combination is present in an amount of about 10 mg/mL to about 40 mg/mL, and wherein the CMC/MCC mixed weight ratio is about 11/89.

26. The pharmaceutical composition of claim 1, wherein the one or more excipient comprises dextrose, and wherein the dextrose is present in an amount of about 10 mg/mL to about 1 g/mL.

27. The pharmaceutical composition of claim 1, wherein the one or more excipient comprises maltodextrin, and wherein the maltodextrin is present in an amount of about 10 mg/mL to about 1 g/mL.

28. The pharmaceutical composition of claim 1, wherein the gastrointestinal surface is the esophageal epithelium.

29. The pharmaceutical composition of claim 1, further comprising a preservative.

30. The pharmaceutical composition of claim 29, wherein the preservative is selected from sodium benzoate, potassium sorbate, or combinations thereof.

31. The pharmaceutical composition of claim 1 further comprising an antioxidant.

32. The pharmaceutical composition of claim 31, wherein the antioxidant is edetate; and wherein edetate is present in an amount of about 0.05 mg/mL to about 25 mg/mL.

33. The pharmaceutical composition of claim 1, further comprising a buffering agent.

34. The pharmaceutical composition of claim 33, wherein the buffering agent comprises citrate; and wherein citrate is present in an amount of about 0.1 mg/mL to about 30 mg/mL.

35. The pharmaceutical composition of claim 1, further comprising a surfactant.

36. The pharmaceutical composition of claim 35, wherein the surfactant comprises polysorbate 80; and wherein the polysorbate 80 is present in an amount of 0.05 mg/mL to about 1 mg/mL.

37. The pharmaceutical composition of claim 1, further comprising a preservative.

38. The pharmaceutical composition of claim 1, wherein the one or more excipient is hydroxyethylcellulose (HEC), hydroxypropylmethyl-cellulose (HPMC), carboxymethyl-cellulose (CMC), microcrystalline cellulose (MCC), carbomer or a combination thereof, and wherein the one or more excipient is present in an amount of about 5 mg/mL to about 100 mg/mL.

39. The pharmaceutical composition of claim 38, wherein the additional excipient is selected from maltodextrin, dextrose and combinations thereof, and wherein the additional excipient is present in an amount of about 1 mg/mL to about 1.5 g/mL.

40. The pharmaceutical composition of claim 1, wherein the pharmaceutical composition has a total volume of about 1 mL to about 20 mL.

41. The pharmaceutical composition of claim 1, wherein a singe dose of the pharmaceutical composition has a total volume of about 1 mL to about 20 mL.

42. The pharmaceutical composition of claim 1, wherein the pharmaceutical composition has a viscosity such that when a single dose of the pharmaceutical composition is orally administered to an individual, the pharmaceutical composition at least partially coats the esophagus and topically delivers a therapeutically effective amount of therapeutic agent to the esophagus.

43. The pharmaceutical composition of claim 1, wherein the pharmaceutical composition has a mucoadhesive characteristic such that when a single dose of the pharmaceutical composition is orally administered to an individual, the pharmaceutical composition adheres to an esophageal surface of the individual for a time sufficient to allow topical delivery of a therapeutically effective amount of the therapeutic agent to the esophagus.

44. A method of treating a gastrointestinal inflammatory disorder, the method comprising orally administering to an individual in need thereof a pharmaceutical composition comprising:
a. a therapeutically effective amount of one or more therapeutic agent;
b. one or more excipient that increases the interaction of the composition with a gastrointestinal surface;
c. optionally, one or more sweetener, one or more flavoring agent, or a combination thereof; and
d. one or more pharmaceutically acceptable carrier, vehicle or a combination thereof;
wherein the oral pharmaceutical composition is physically and chemically stable; and wherein the oral pharmaceutical composition is topically active.

45. The method of claim 44, wherein the gastrointestinal disorder is selected from gastrointestinal inflammation, gastrointestinal cancer, gastrointestinal infection, gastrointestinal motility dysfunction, or lesions, wounds or contusions of tissue of a gastrointestinal surface.

46. The method of claim 45, wherein the gastrointestinal inflammation is esophageal inflammation.

47. The method of claim 46, wherein the gastrointestinal inflammation is eosinophilic esophagitis, intermediate esophagitis, epithelial hyperplasia, basal cell hyperplasia, elongated papillae, dilated vessels in papillae, fungal esophagitis, viral esophagitis, bacterial esophagitis, corrosive esophagitis, radiation esophagitis, chemotherapy esophagitis, graft vs. host disease, a skin disease with esophageal involvement, bullous pemphigoid, pemphigus vulgaris, epidermolysis bollosa, Stevens-Johnson syndrome, Behçet's disease, sarcoidosis, idiopathic esophagitis, eosinophilic gastritis, Ménétrier's disease, parasitic gastritis, lymphocytic esophagitis, inflammatory bowel disease-associated esophagitis, parasitic gastritis, lymphocytic esophagitis, inflammatory bowel disease-associated esophagitis, tracheoesophageal fistulae, an inflammatory bowel disease involving the esophagus, reflux esophagitis, Crohn's disease, proximal gastrointestinal pathology, eosinophilic gastrointestinal inflammation, celiac disease, eosinophilic duodenitis, duodenal eosinophilia, functional dyspepsia, esophageal inflammation secondary to caustic/irritant ingestion, persistent/recurrent esophageal strictures of any cause and including caustic/irritant ingestion, pill-induced esophagitis, systemic diseases, congenital diseases, post-surgery inflammation or gastro enteritis.

48. The method of claim 47, wherein the individual has eosinophilic esophagitis.

49. The method of claim 46, wherein the individual has been diagnosed with reflux esophagitis, gastroesophageal reflux disease (GERD), nonerosive reflux disease (NERD), Barrett's Esophagus or erosive esophagitis.

50. The method of claim 45, wherein the gastrointestinal cancer is esophageal cancer.

51. The method of claim 45, wherein the gastrointestinal infection is a bacterial infection or a fungal infection.

52. The method of claim 45, wherein the gastrointestinal motility dysfunction is esophageal motility dysfunction.

53. The method of claim 45, wherein the lesions, wounds or contusions of tissue of a gastrointestinal surface are lesions, wounds or contusions of the esophageal epithelium.

54. The method of claim 44, wherein the therapeutically effective agent is administered in a concentration of about 0.0001 mg/mL to about 10 mg/mL per cm of esophageal length of the individual.

55. A kit comprising a multiple unit container and a plurality of doses of an oral pharmaceutical composition, wherein each dose of the pharmaceutical composition comprises:
a. a therapeutically effective amount of a topically active therapeutic agent;
b. one or more excipient that increases the interaction of the composition with a gastrointestinal surface;
c. optionally one or more sweetener, one or more flavoring agent, or a combination thereof; and
d. one or more pharmaceutically acceptable carrier, vehicle or a combination thereof;
wherein the oral pharmaceutical composition is physically and chemically stable; and wherein the oral pharmaceutical composition is topically active

56. The kit of claim 55, wherein each dose of the pharmaceutical composition further comprises a chelating agent, a surfactant, a buffering agent, and a flavoring agent.

57. The kit of claim 55, wherein the vehicle is a liquid vehicle comprising an aqueous medium.

58. The kit of claim 55, wherein the at least one additional excipient comprises one or more maltodextrin, dextrose, hydroxyethylcellulose (HEC), hydroxypropylmethyl-cellulose (HPMC), carboxymethyl-cellulose (CMC), microcrystalline cellulose (MCC), carbomer or combinations thereof.

59. The kit of any of claim 55, wherein the kit comprises about 10 to about 60 doses of the pharmaceutical composition.

60. The kit of any of claim 55, wherein the kit comprises about 330 mL of the pharmaceutical composition.

61. The kit of claim 55, wherein the kit comprises about 55 mL of the stable pharmaceutical composition.

62. The kit of claim 55, wherein the kit further comprises a metering device for administering the composition to an individual.

63. The kit of claim 62, wherein the metering device is incorporated into the multiple unit container.
